(19) United States
(12) Patent Application Publication
(10) Pub. No.: US 2026/0140083 A1
(43) Pub. Date: May 21, 2026

(54) Regenerative Plant Sensor

(71) Applicants: BOARD OF REGENTS, THE UNIVERSITY OF TEXAS SYSTEM, Austin, TX (US); THE UNITED STATES OF AMERICA, AS REPRESENTED BY THE SECRETARY OF AGRICULTURE, Washington, DC (US)

(72) Inventors: Shawana Tabassum, Tyler, TX (US); Md Sariful Islam, Wellington, FL (US); James Todd, Houma, LA (US); Madhavi Pali, Tyler, TX (US); Najmul Islam, Tyler, TX (US)

(73) Assignees: BOARD OF REGENTS, THE UNIVERSITY OF TEXAS SYSTEM, Austin, TX (US); THE UNITED STATES OF AMERICA, AS REPRESENTED BY THE SECRETARY OF AGRICULTURE, Washington, DC (US)

(21) Appl. No.: 19/327,540

(22) Filed: Sep. 12, 2025

Related U.S. Application Data
(60) Provisional application No. 63/720,980, filed on Nov. 15, 2024.

(51) Int. Cl. G01N 27/327; G01N 27/416; G01N 33/53

(52) U.S. Cl. CPC G01N 27/3275; G01N 27/416; G01N 33/5308; G01N 2333/43

(57) ABSTRACT

A regenerative plant sensor and method for in situ monitoring of analytes in plant sap are disclosed. The sensor includes a substrate embedded with one or more shape memory alloy (SMA) wires, a sensor layer, a microfluidic system in communication with a fluid chamber and the sensor layer, one or more pumps in communication with the microfluidic system, a microcontroller in electrical communication with the sensor layer, the one or more pumps, and one or more SMA wires, wherein one or more SMA wires form a fluid gate in the flexible substrate, and wherein the fluid chamber is filled with a regeneration solution. Data are processed by an integrated data acquisition system, facilitating real-time data access for monitoring plant analytes. Also disclosed are methods for positioning the sensor on plants, regulating sap flow into the sensor, measuring analyte concentrations, and regenerating the sensor layer for subsequent measurement cycles.

### CROSS REFERENCE TO RELATED APPLICATION

This application claims priority benefit to U.S. Provisional application No. 63/720,980, filed Nov. 15, 2024, the disclosure of which is incorporated herein by reference in its entirety.

### STATEMENT REGARDING FEDERALLY SPONSORED RESEARCH OR DEVELOPMENT

This invention was made with Federal government support under USDA-ARS 58-6030-3-005 awarded by the United States Department of Agriculture. The government has certain rights in the invention.

### COLOR DRAWINGS

The patent or application file contains at least one drawing executed in color. Copies of this patent or patent application publication with color drawing(s) will be provided by the Office upon request and payment of the necessary fec.

### TECHNICAL FIELD

An integrated sensor suite capable of non-destructive and real-time measurements of plant analytes.

### BACKGROUND

It is crucial to determine the optimum maturity of sugarcane in order to harvest the cane at the appropriate age. Harvesting under-aged or over-aged cane leads to losses in cane sugar yield, problems in sugar recovery, and poor juice quality. Immature sugarcane has a high content of reducing sugars and color precursor compounds, producing juice with a darker color. Peak maturation is defined by sucrose accumulation with reductions in the content of reducing sugars (primarily glucose and fructose). Hence, it is imperative to know the optimum time to harvest sugarcane.

The maturity of sugarcane is currently determined by visually inspecting the field and measuring the Brix using a hand refractometer on randomly selected plants. The indicators of optimum maturity highly depend on cultivars and the local environment. This method is complex, time and labor-intensive, incurs destructive sample collection, and lacks real-time information on sucrose levels. Although refractometry provides real-time information at a lower cost, the process relies on destructive juice sampling from the plant. The aforementioned processes involve discrete measurements, lack of automation, and lack of remote monitoring, and hence are not robust and efficient for sampling a large number of plants. Plants are selected randomly in each block for sample collection and the average sucrose content per block is assessed. Currently, sugar monitoring at the plant scale is impossible due to the lack of automation and scalability. It is also documented that the selection process in the sugarcane breeding program has faced many challenges in terms of labor, time, and accurate measurements of sucrose content in the progeny.

Moreover, climatic variables and nitrogen (N) fertilization substantially impact the growth, yield, and quality of sugar in sugarcane. Frequent drought conditions negatively impact sucrose yield, especially in the sandy soils in areas such as Florida. Water stress also alters plant physiology, i.e., photosynthesis, respiration, and stomatal conductance. In addition, low N availability is one of the major limiting factors for sugarcane grown on sand soil and marginal land. However, N-fertilization is poorly managed and N-use efficiency continues to decline each year. Currently, the N recommendations for sugarcane are based on expected sugarcane yields, which may result in the excessive application of N leading to soil N unbalance. Therefore, it is imperative to measure N uptake in real-time during the growth period to determine optimum N rates in sugarcane and avoid the unnecessary application of N fertilizers and reduce the cost of production.

Thus, there exists a need for real-time, in situ sensor technology to alleviate this problem by measuring the sucrose content continuously and non-destructively, so that breeders can select high sucrose clones with high confidence, which may in turn reduce the field sampling work.

### SUMMARY

The present disclosure provides regenerative, real-time and in situ monitoring of sucrose in plants that allows for the detection of the spatial and temporal distribution of sugar content directly in the cane stalk, capability of sensor regeneration for extended lifespan, wireless data transfer capability, and low-cost solution while incurring minimal damage to the plant and avoiding the destructive sample collection procedure. Provided herein is a regenerative plant sensor for monitoring analytes, including sucrose, within the sap of a plant, comprising a flexible substrate embedded with one or more shape memory alloy (SMA) wires, a substrate comprising a sensor layer, the sensor layer comprising a working electrode (WE), a counter electrode (CE), and a reference electrode (RE), wherein one or more electrodes on said sensor layer are functionalized with one or more of a first metal, a second metal, and a composition comprising a synthetic polymer and a hydrogel. In some aspects, the regenerative plant sensor comprises a regeneration system comprising a microfluidic system in communication with a fluid chamber and the sensor layer, one or more pumps in communication with the microfluidic system, a microcontroller in electrical communication with the sensor layer, the one or more pumps, and the one or more shape memory alloy (SMA) wires, wherein the one or more SMA wires form a fluid gate in the flexible substrate, and wherein the fluid chamber is filled with a regeneration solution. The sensor layer is constructed from a metal-coated composite of polymer and hydrogel, and can detect analytes such as sugars, including sucrose, and hormones directly from the sap. This configuration allows for accurate, real-time measurement of specific analytes. The regeneration system, which is connected to and a part of the sensor, allows a regeneration solution to flow through a microfluidic system in communication with a fluid chamber and the sensor layer, wherein one or more pumps in communication with the microfluidic system move the solution throughout the sensor to wash the analyte, and subsequently remove the solution to allow for repeated testing of the analyte.

In some aspects, the regenerative plant sensor comprises a data acquisition system integrated within the regenerative plant sensor to process and transmit the data collected from the sensor. The data acquisition system comprises a processor, a communication unit, and a memory unit that stores software instructions for controlling data collection and communication. Through said communication unit, the processed data can be sent to external devices, including cloud-based IoT servers, enabling remote monitoring and real-time data sharing.

Provided herein is a regenerative plant sensor to measure analytes in plant sap. The sensor is regenerative and includes a microfluidic network connected to a fluid chamber containing a regeneration solution. The regeneration solution, stored in the chamber, is pumped through the microfluidic system to periodically cleanse and refresh the sensor layer or assay, ensuring continued accuracy and longevity of the measurements. The SMA wire gate can be comprised of nitinol or similar material to enable one-way and two-way actuation, allowing the system to control fluid access to the sensor layer with precision.

Provided herein is a method for using the regenerative plant sensor to measure analytes in plant sap in a non-destructive manner. The method involves placing the sensor on a plant, after which a first electrical potential is applied to open the SMA fluid gate, allowing sap to infiltrate the sensor layer. After measurement, a second electrical potential is applied to close the SMA fluid gate. The sensor layer is then regenerated using the regeneration processes described herein, whereby a regeneration solution is pumped over said sensor layer, and the solution is subsequently removed to complete a regeneration cycle. This method enables efficient, repeated, non-destructive analyte detection cycles, enhancing the sensor's utility for continuous monitoring.

In some aspects, the regenerative plant sensor provides real-time measurements of analytes like sucrose to determine crop maturity. The regenerative plant sensor may provide for detection of sugar cane readiness for harvest. The regenerative plant sensor, constructed with a substrate comprising a sensor layer, conforms to the surface of the plant, making it suitable for a wide range of agricultural applications. In some aspects, the substrate comprising the sensor layer is a 3D printed substrate. The low-power draw of the measurement, regeneration, and data transmittal processes further allow the system to complete multiple measurement cycles on a single power supply, facilitating extended field deployment.

### BRIEF DESCRIPTION OF THE DRAWINGS

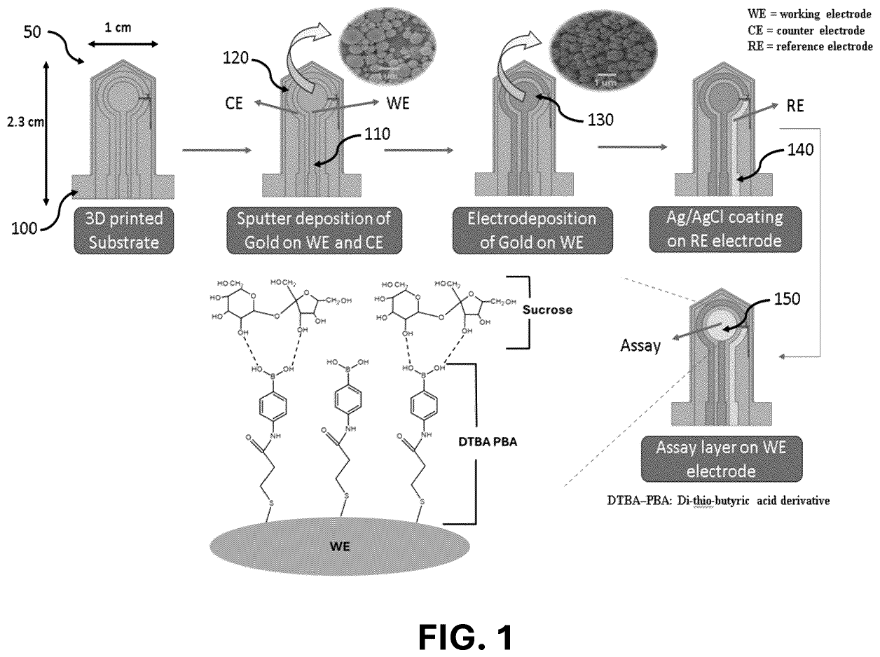

FIG. 1 shows the fabrication of the regenerative plant sensor, including the formation of the gold sputter deposition layer, the gold electrodeposition layer, the silver/silver chloride layer, and the assay layer (collectively, the “sensor layer”).

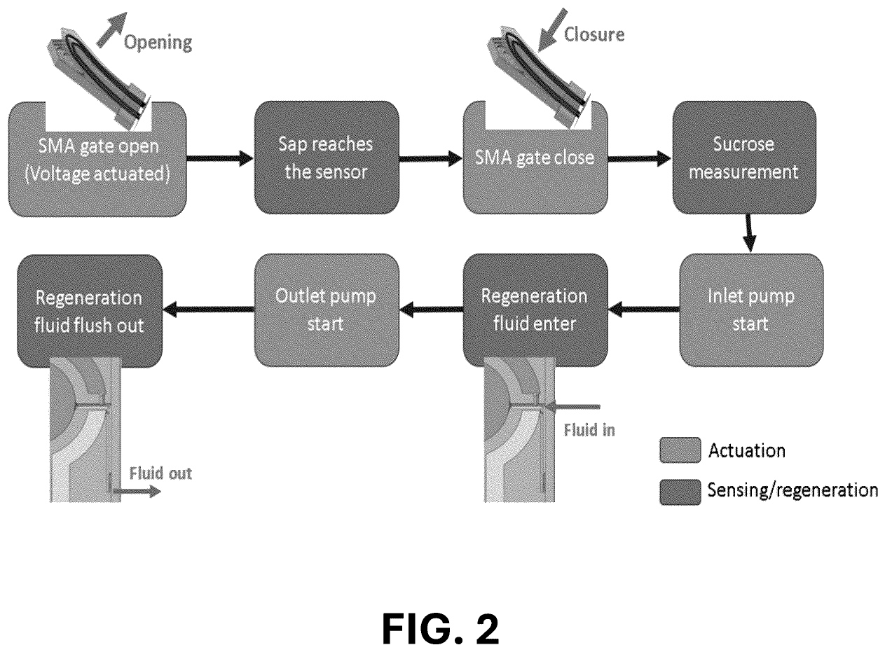

FIG. 2 shows a flowchart of the process by which the regenerative plant sensor (a) senses analyte concentration and (b) undergoes the regeneration process to allow for repeated use.

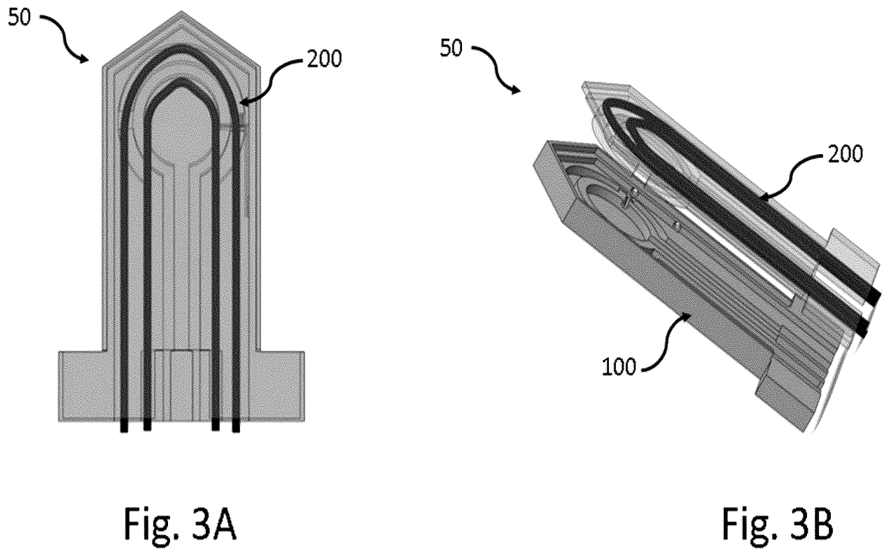

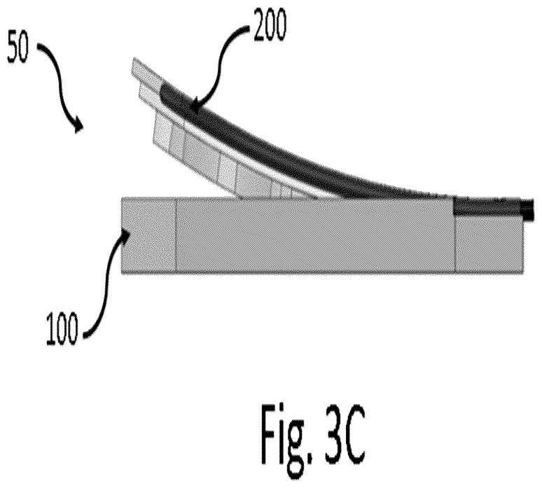

FIGS. 3A-3C show an embodiment of the regenerative plant sensor. FIG. 3A shows a front view of the sensor including a flexible substrate embedded with one or more shape memory alloy (SMA) wires forming the SMA wire gate, and the portions of the sensor for the electrodes on the substrate comprising a sensor layer. FIG. 3B shows an exploded side view of the sensor including the shape memory alloy (SMA) wire gate (top) and substrate comprising a sensor layer (bottom). FIG. 3C shows a side view of the sensor wherein the SMA wire gate is activated in the “open” position.

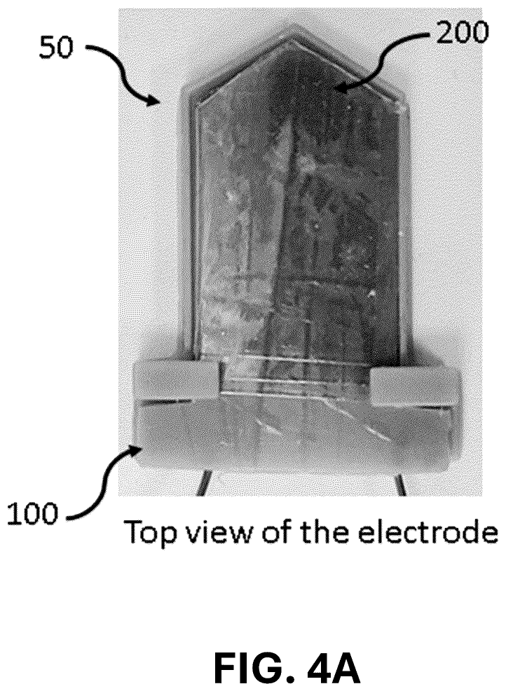

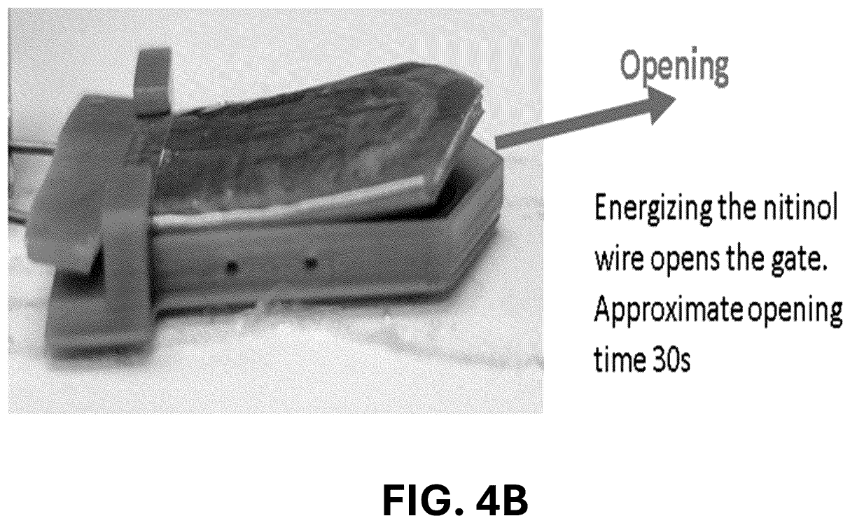

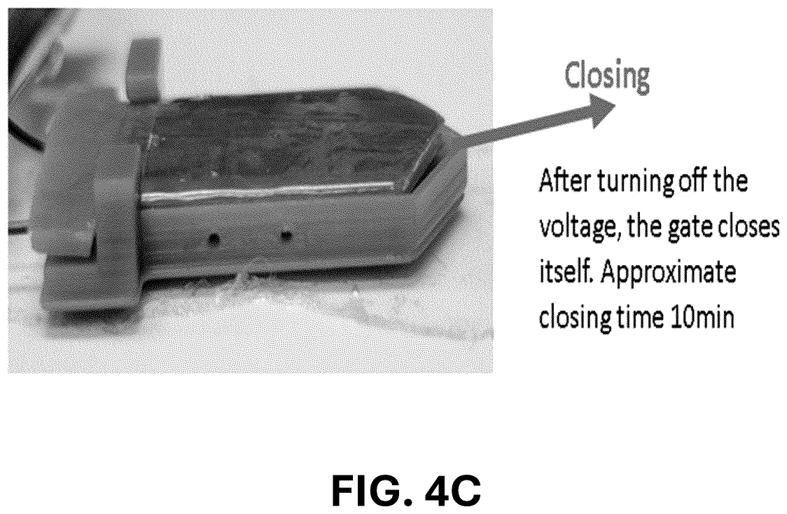

FIGS. 4A-4C show an embodiment of the sensor including a 2-way SMA wire gate. FIG. 4A shows a front view of the sensor. FIG. 4B shows a side view of the sensor wherein the SMA wire gate is open after the SMA wire is energized. FIG. 4C shows a side view of the sensor wherein the SMA wire gate is closed after voltage is disconnected.

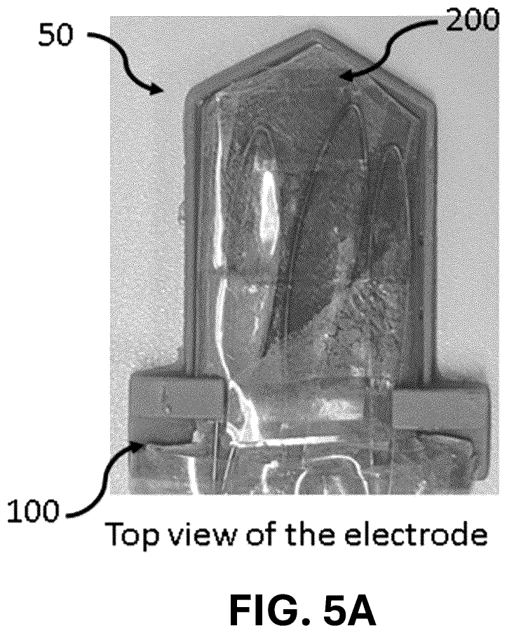

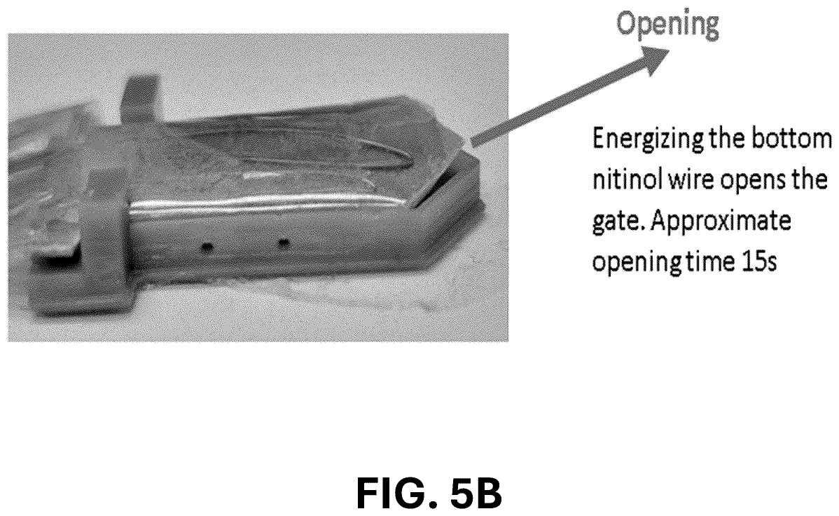

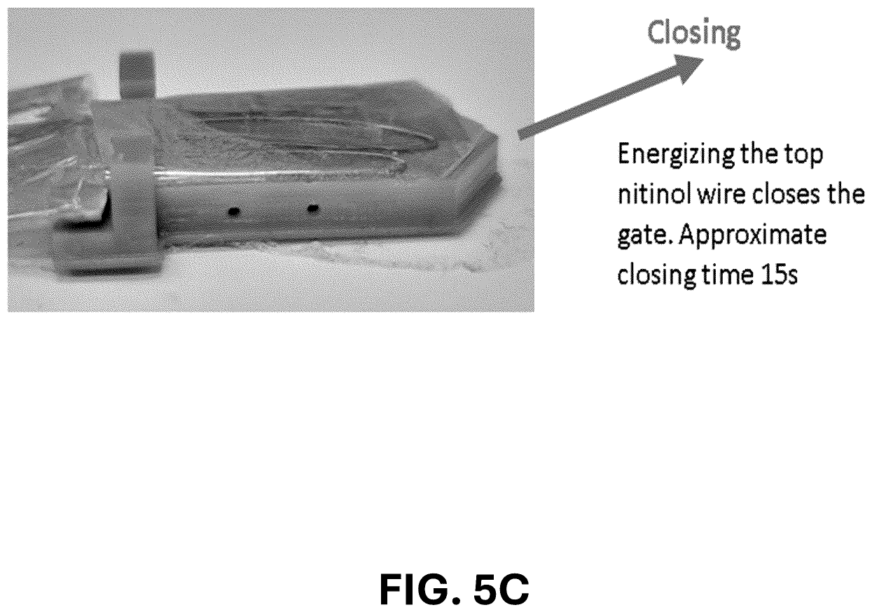

FIGS. 5A-5C show an embodiment of the sensor including a 2-way SMA wire gate. FIG. 5A shows a front view of the sensor. FIG. 5B shows a side view of the sensor wherein the SMA wire gate is open after energizing a bottom SMA nitinol wire. FIG. 5C shows a side view of the sensor wherein the SMA wire gate is closed after the top SMA wire is energized.

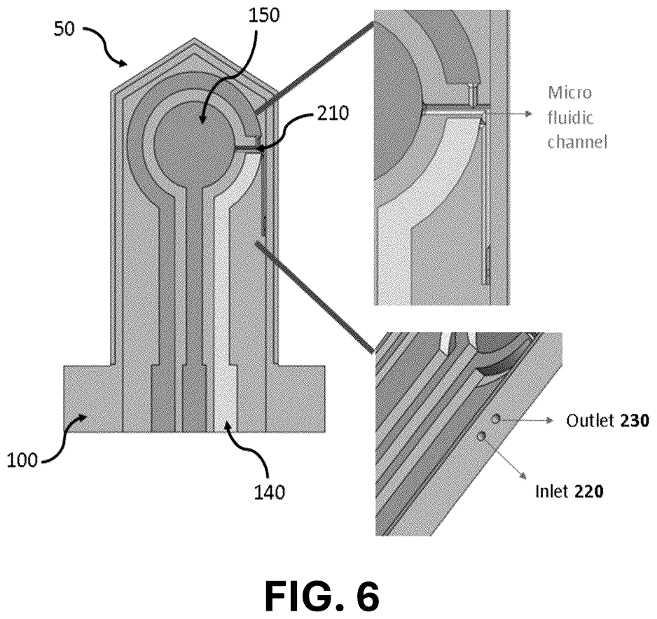

FIG. 6 shows a schematic of an embodiment of the sensor including a micro fluidic channel including a fluid inlet and outlet.

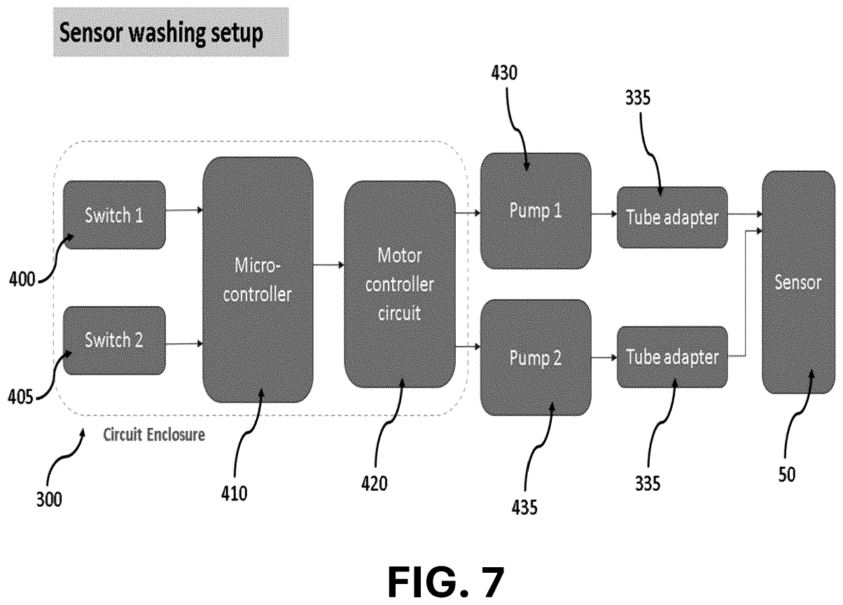

FIG. 7 shows a flowchart of the sensor washing process, wherein a first switch (“Switch 1”), a second switch (“Switch 2”), a microcontroller, and a motor controller circuit are encompassed within a circuit enclosure.

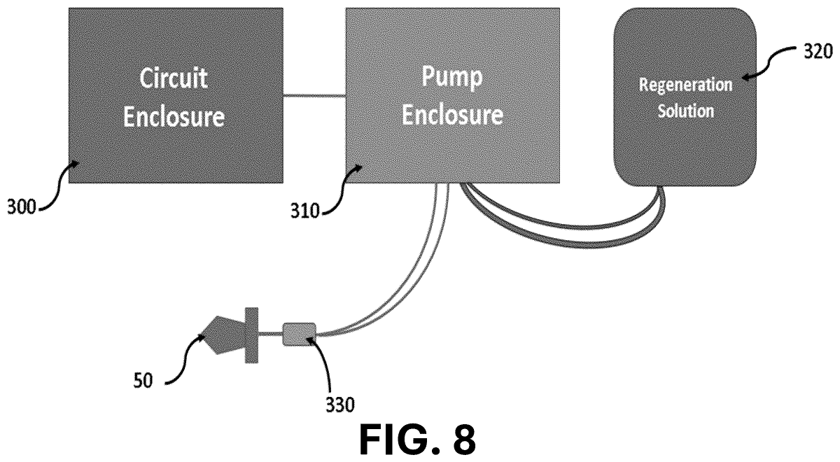

FIG. 8 shows an overview of the sensor washing setup, including the circuit enclosure, pump enclosure, regeneration solution, tube adaptor, and regenerative plant sensor (sucrose sensor).

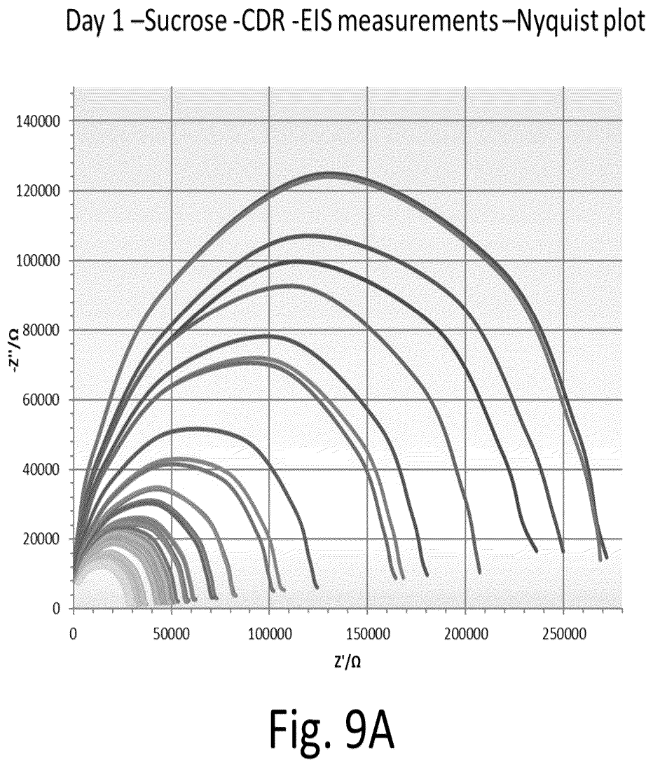

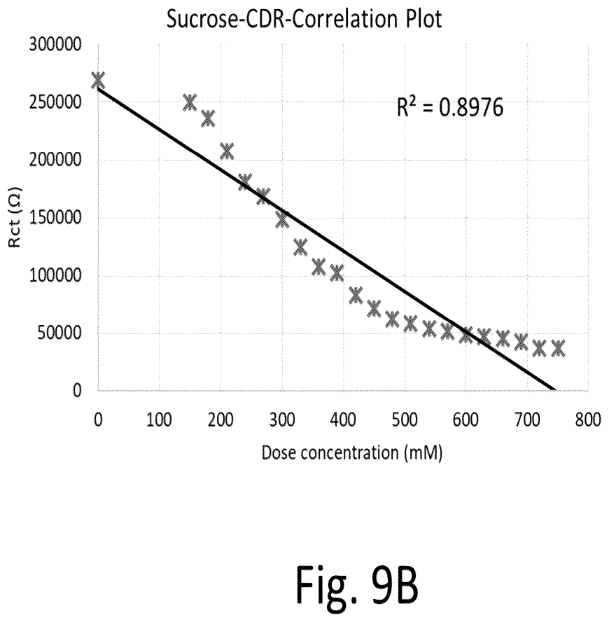

FIGS. 9A-9B show the Sucrose CDR/EIS measurements for Day 1. FIG. 9A shows the Nyquist plot, showing the imaginary (Z″) versus the real impedance (Z′). FIG. 9B shows the scatter plot of Rct versus sucrose dose, wherein a straight line is fitted through the scattered Rct values to get the CDR correlation plot.

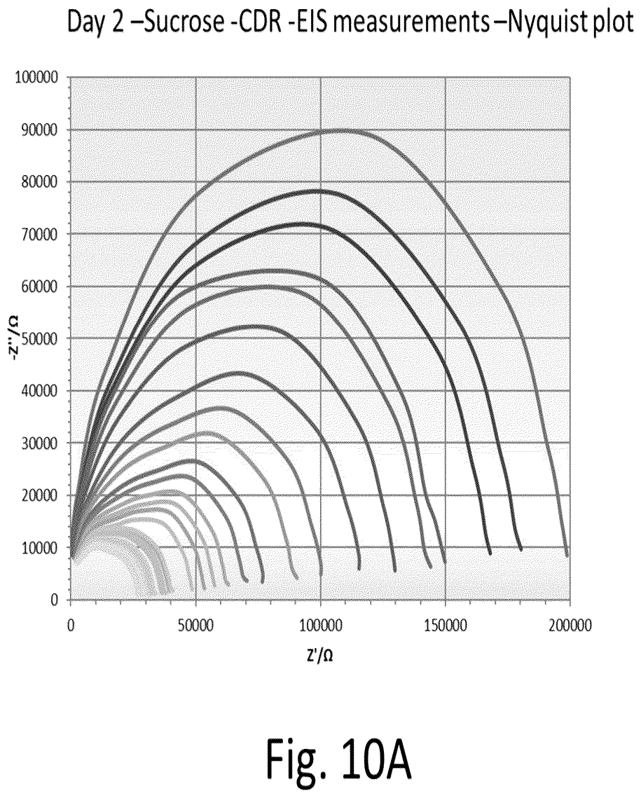

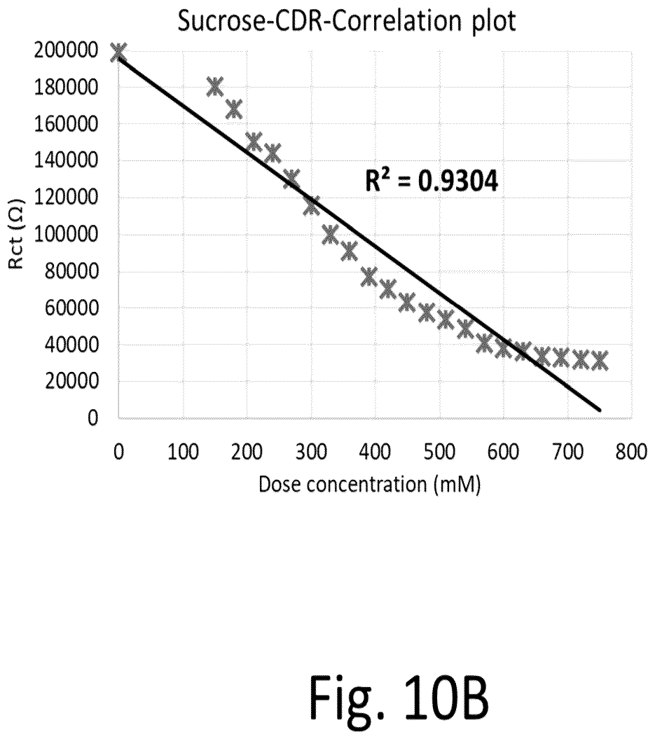

FIGS. 10A-10B show the Sucrose CDR/EIS measurements for Day 2. FIG. 10A shows the Nyquist plot, showing the imaginary (Z″) versus the real impedance (Z′). FIG. 10B shows the scatter plot of Rct versus sucrose dose, wherein a straight line is fitted through the scattered Rct values to get the CDR correlation plot.

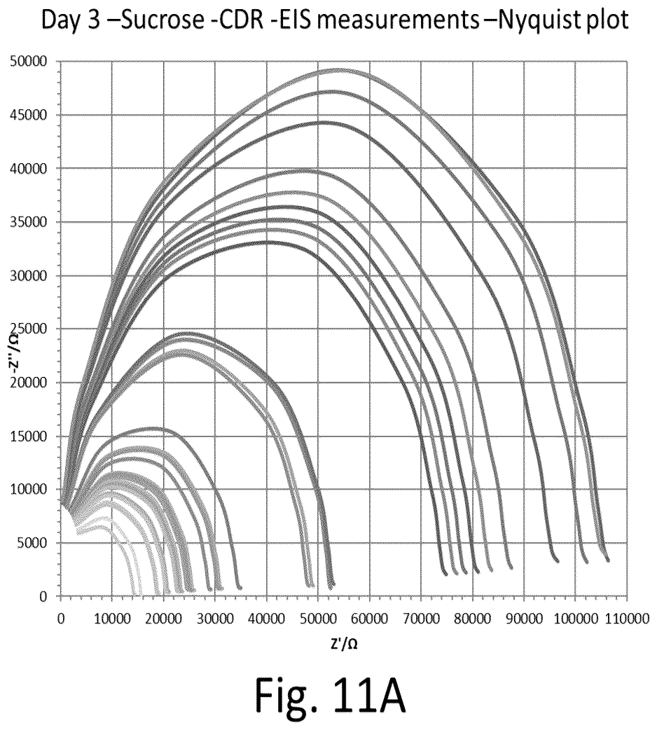

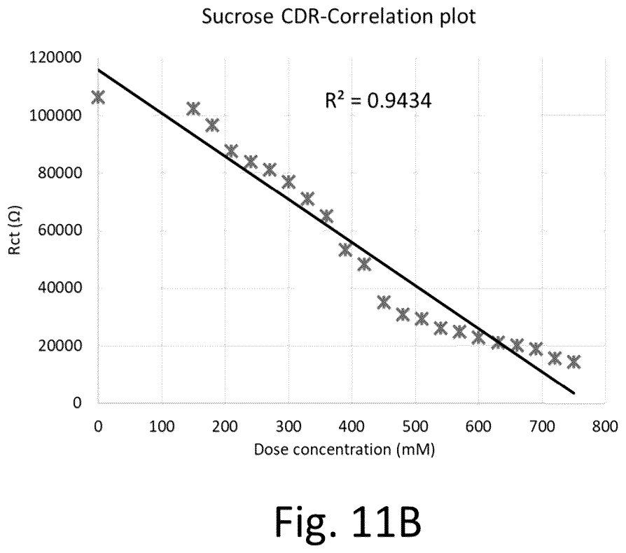

FIGS. 11A-11B show the Sucrose CDR/EIS measurements for Day 3. FIG. 11A shows the Nyquist plot, showing the imaginary (Z″) versus the real impedance (Z′). FIG. 11B shows the scatter plot of Rct versus sucrose dose, wherein a straight line is fitted through the scattered Rct values to get the CDR correlation plot.

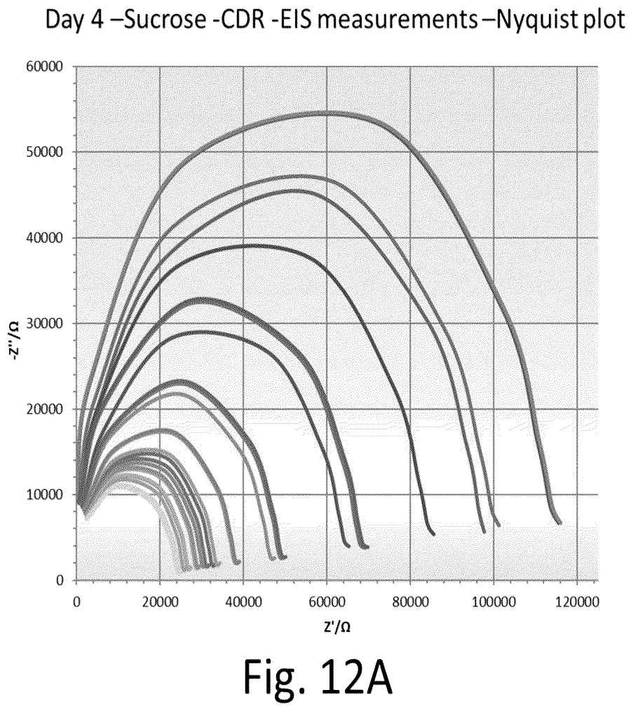

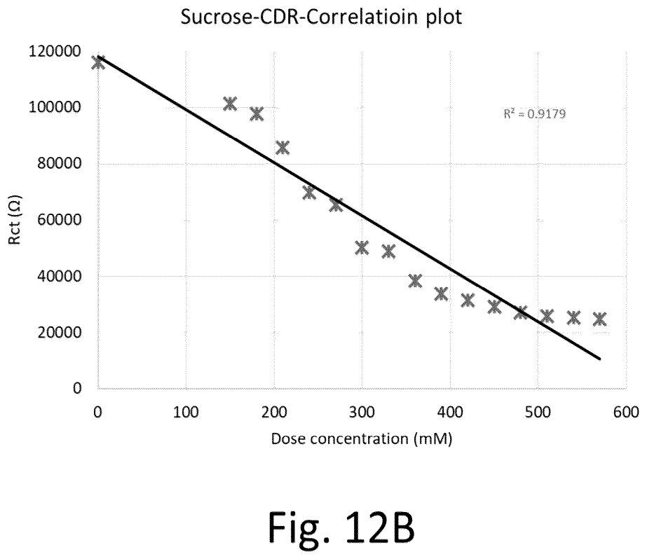

FIGS. 12A-12B show the Sucrose CDR/EIS measurements for Day 4. FIG. 12A shows the Nyquist plot, showing the imaginary (Z″) versus the real impedance (Z′). FIG. 12B shows the scatter plot of Rct versus sucrose dose, wherein a straight line is fitted through the scattered Rct values to get the CDR correlation plot.

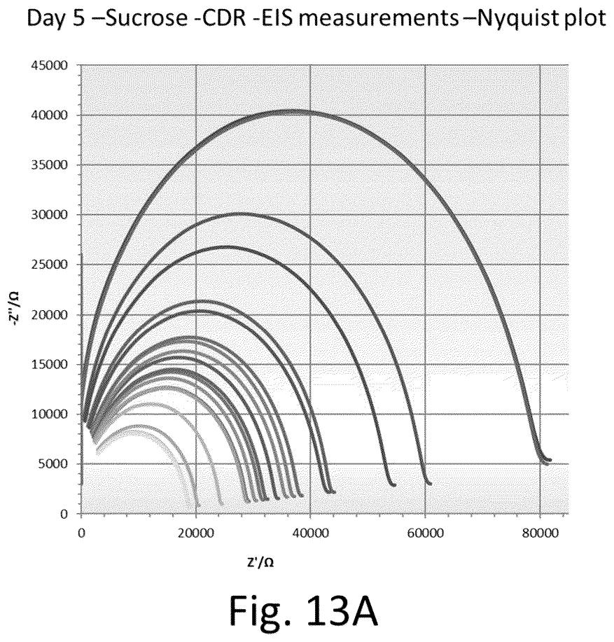

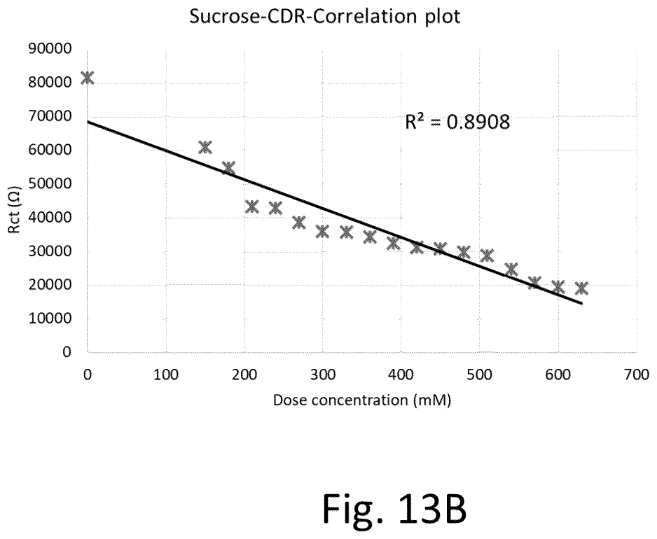

FIGS. 13A-13B show the Sucrose CDR/EIS measurements for Day 5. FIG. 13A shows the Nyquist plot, showing the imaginary (Z″) versus the real impedance (Z′). FIG. 13B shows the scatter plot of Rct versus sucrose dose, wherein a straight line is fitted through the scattered Rct values to get the CDR correlation plot.

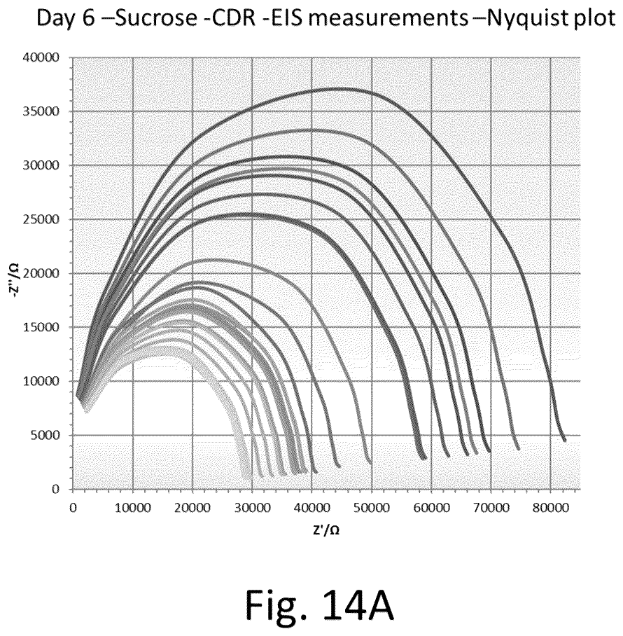

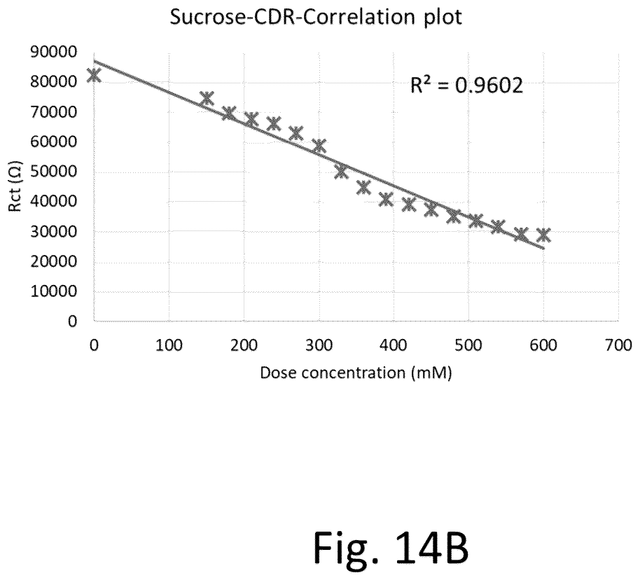

FIGS. 14A-14B show the Sucrose CDR/EIS measurements for Day 6. FIG. 14A shows the Nyquist plot, showing the imaginary (Z″) versus the real impedance (Z′). FIG. 14B shows the scatter plot of Rct versus sucrose dose, wherein a straight line is fitted through the scattered Rct values to get the CDR correlation plot.

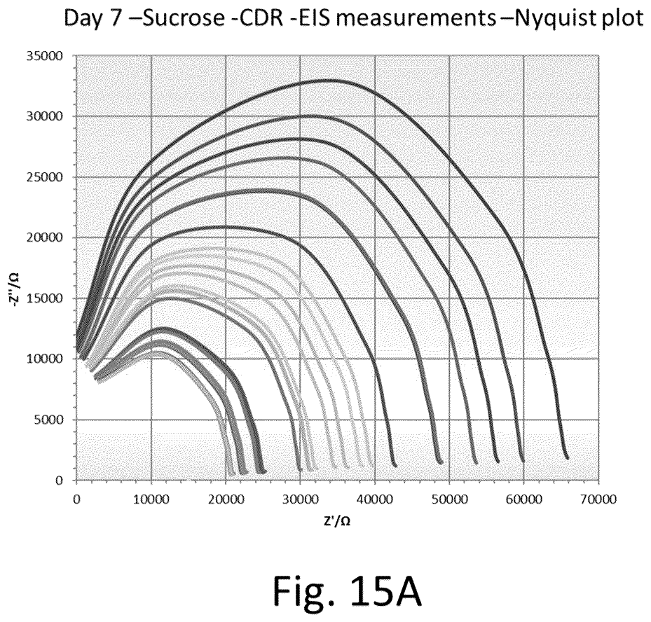

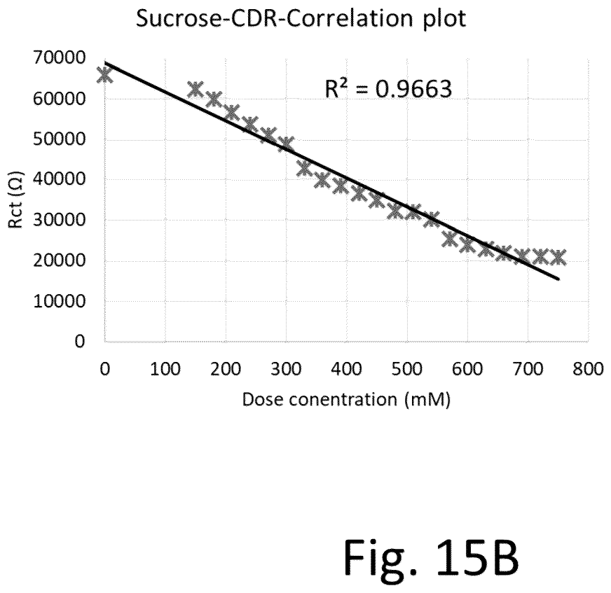

FIGS. 15A-15B show the Sucrose CDR/EIS measurements for Day 7. FIG. 15A shows the Nyquist plot, showing the imaginary (Z″) versus the real impedance (Z′). FIG. 15B shows the scatter plot of Rct versus sucrose dose, wherein a straight line is fitted through the scattered Rct values to get the CDR correlation plot.

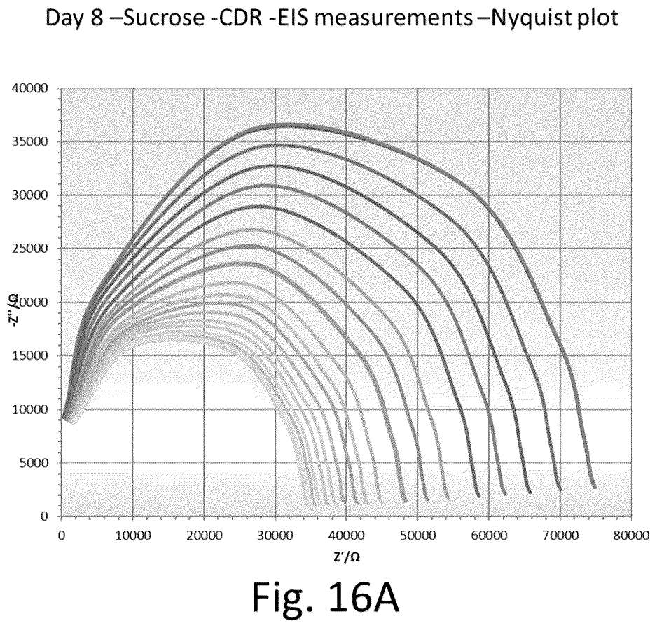

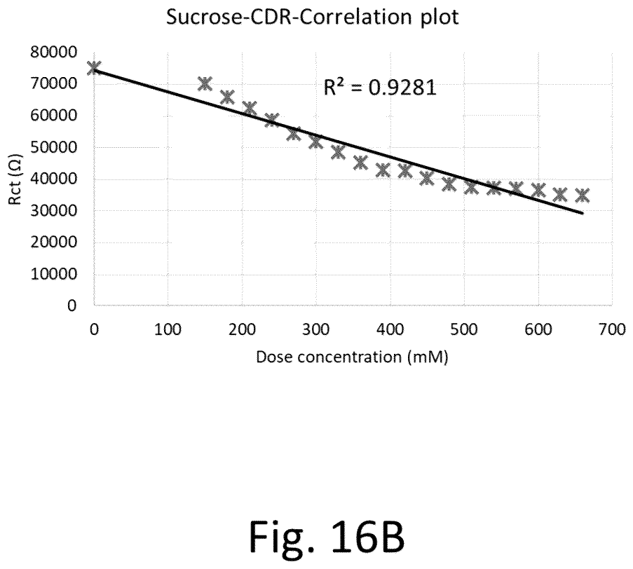

FIGS. 16A-16B show the Sucrose CDR/EIS measurements for Day 8. FIG. 16A shows the Nyquist plot, showing the imaginary (Z″) versus the real impedance (Z′). FIG. 16B shows the scatter plot of Rct versus sucrose dose, wherein a straight line is fitted through the scattered Rct values to get the CDR correlation plot.

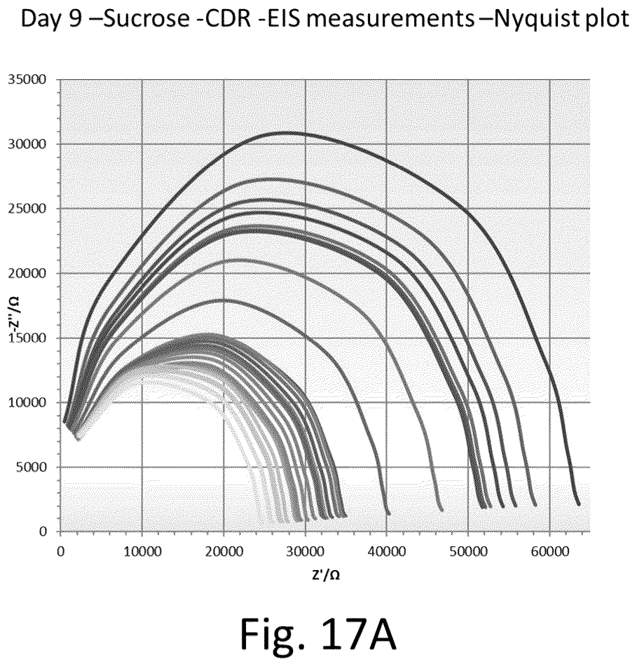

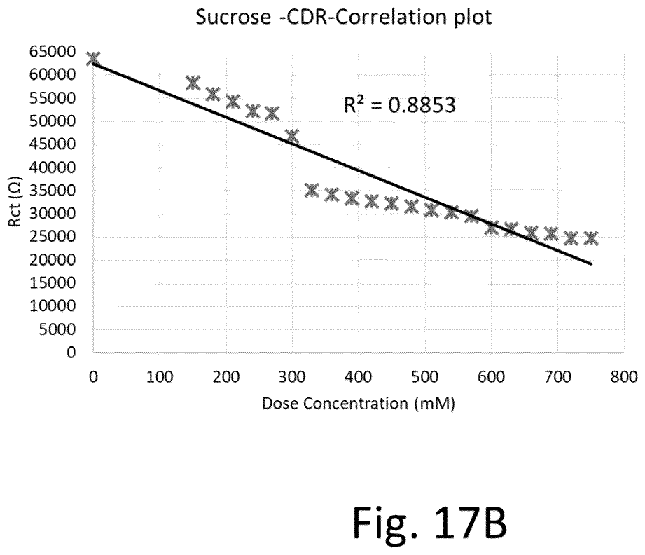

FIGS. 17A-17B show the Sucrose CDR/EIS measurements for Day 9. FIG. 17A shows the Nyquist plot, showing the imaginary (Z″) versus the real impedance (Z′). FIG. 17B shows the scatter plot of Rct versus sucrose dose, wherein a straight line is fitted through the scattered Rct values to get the CDR correlation plot.

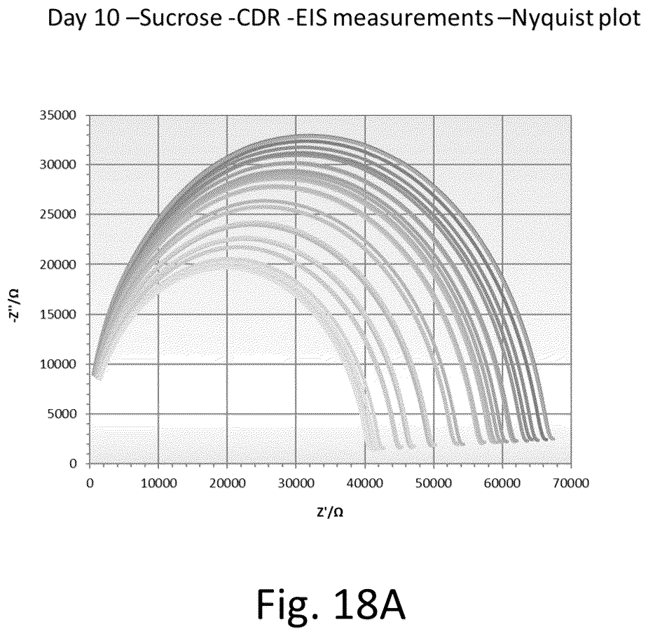

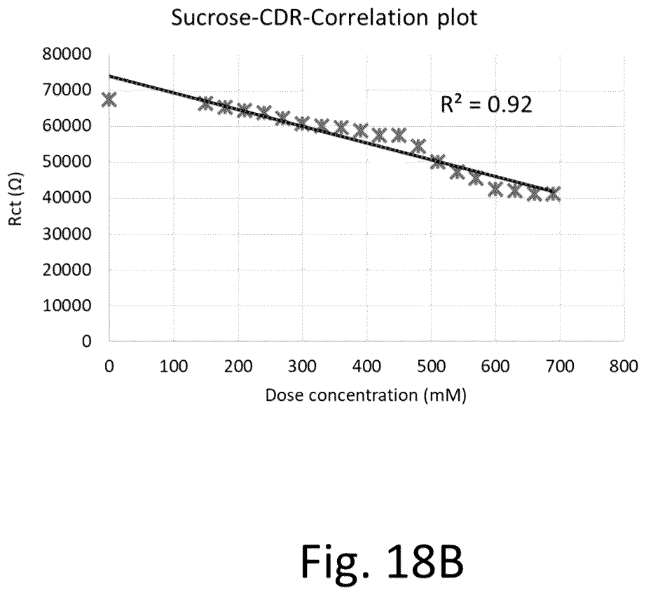

FIGS. 18A-18B show the Sucrose CDR/EIS measurements for Day 10. FIG. 18A shows the Nyquist plot, showing the imaginary (Z″) versus the real impedance (Z′). FIG. 18B shows the scatter plot of Rct versus sucrose dose, wherein a straight line is fitted through the scattered Rct values to get the CDR correlation plot.

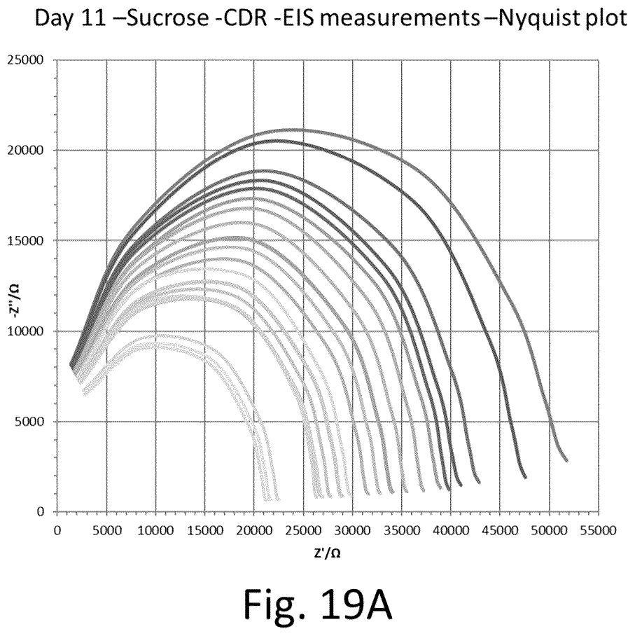

FIGS. 19A-19B show the Sucrose CDR/EIS measurements for Day 11. FIG. 19A shows the Nyquist plot, showing the imaginary (Z″) versus the real impedance (Z′). FIG. 19B shows the scatter plot of Rct versus sucrose dose, wherein a straight line is fitted through the scattered Rct values to get the CDR correlation plot.

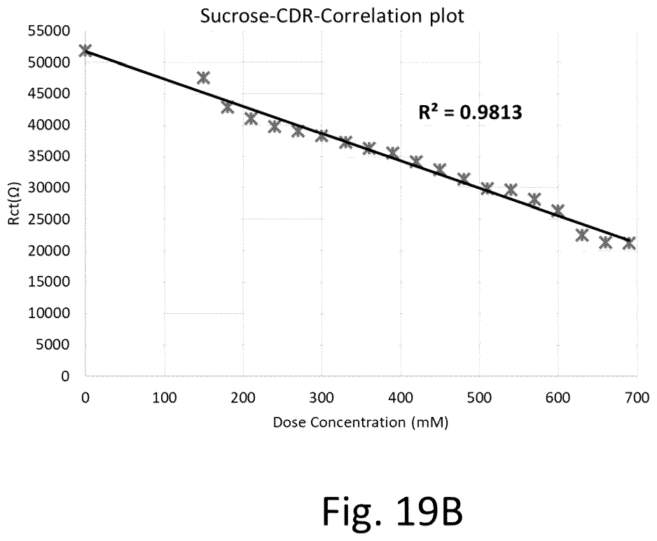

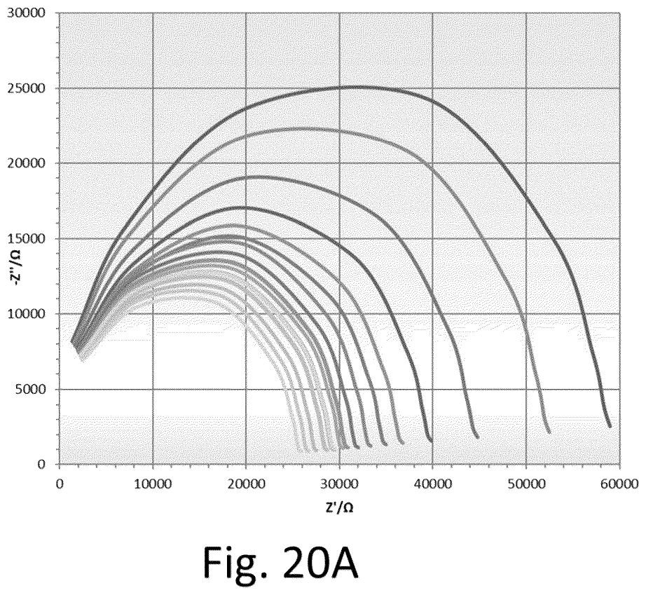

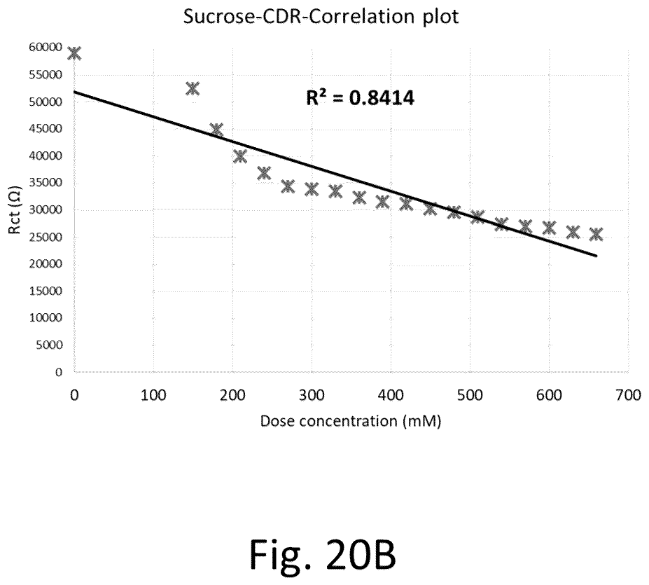

FIGS. 20A-20B show the Sucrose CDR/EIS measurements for Day 12. FIG. 20A shows the Nyquist plot, showing the imaginary (Z″) versus the real impedance (Z′). FIG. 20B shows the scatter plot of Rct versus sucrose dose, wherein a straight line is fitted through the scattered Rct values to get the CDR correlation plot.

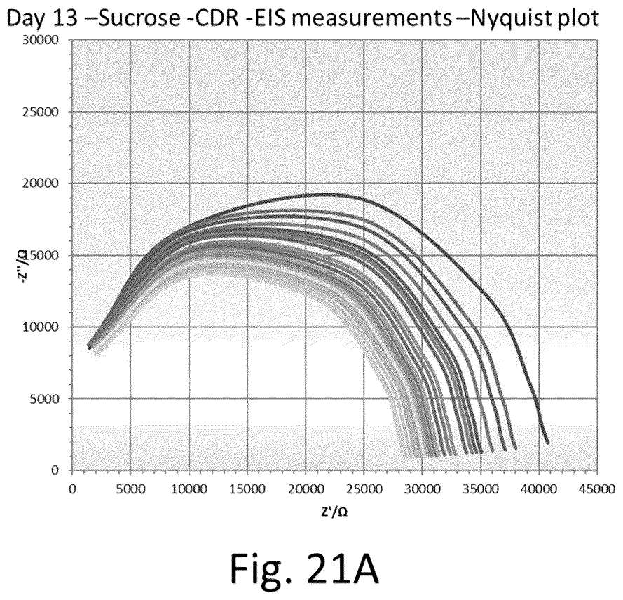

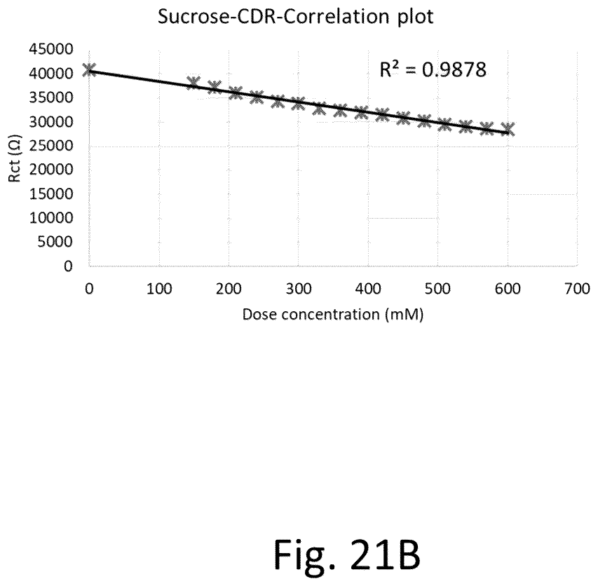

FIGS. 21A-21B show the Sucrose CDR/EIS measurements for Day 13. FIG. 21A shows the Nyquist plot, showing the imaginary (Z″) versus the real impedance (Z′). FIG. 21B shows the scatter plot of Rct versus sucrose dose, wherein a straight line is fitted through the scattered Rct values to get the CDR correlation plot.

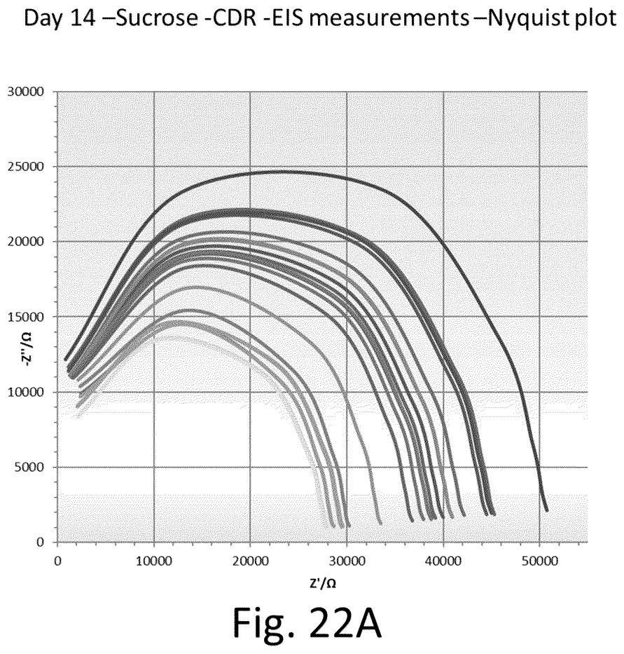

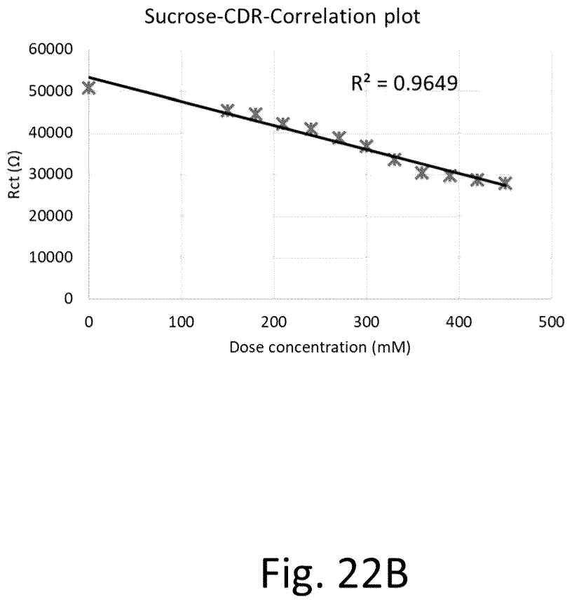

FIGS. 22A-22B show the Sucrose CDR/EIS measurements for Day 14. FIG. 22A shows the Nyquist plot, showing the imaginary (Z″) versus the real impedance (Z′). FIG. 22B shows the scatter plot of Rct versus sucrose dose, wherein a straight line is fitted through the scattered Rct values to get the CDR correlation plot.

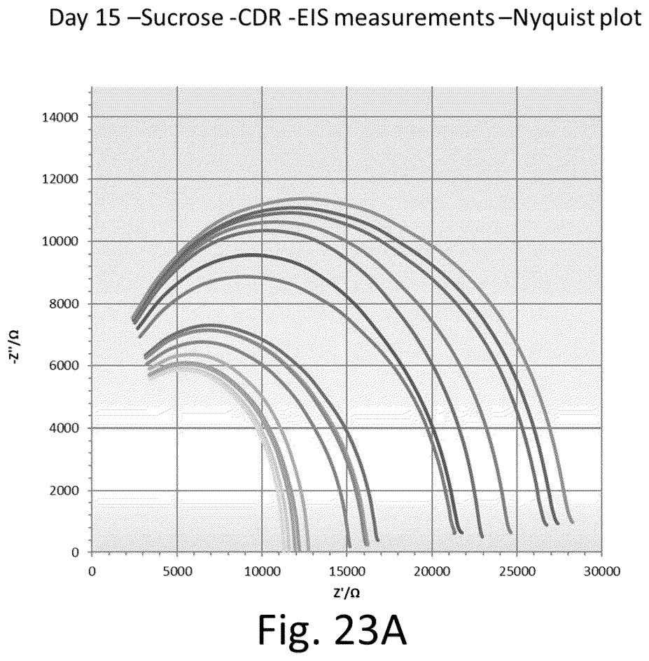

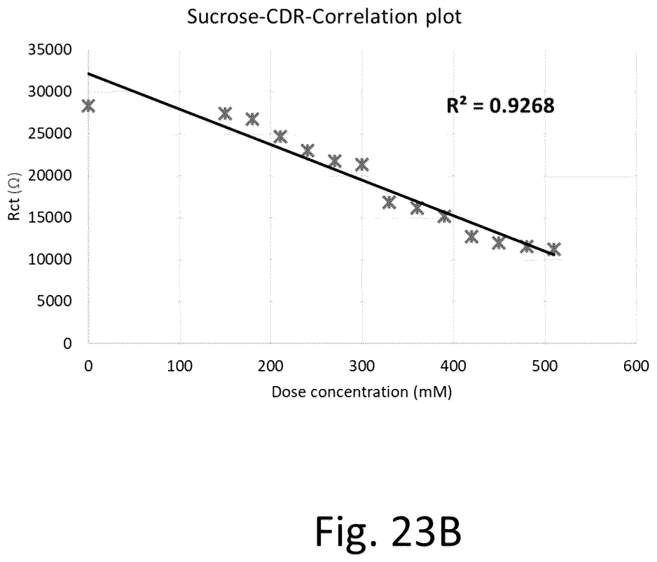

FIGS. 23A-23B show the Sucrose CDR/EIS measurements for Day 15. FIG. 23A shows the Nyquist plot, showing the imaginary (Z″) versus the real impedance (Z′). FIG. 23B shows the scatter plot of Rct versus sucrose dose, wherein a straight line is fitted through the scattered Rct values to get the CDR correlation plot.

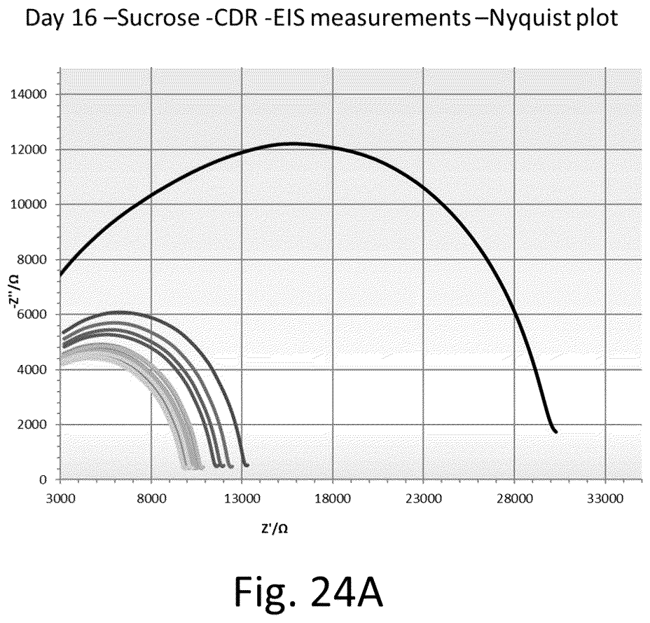

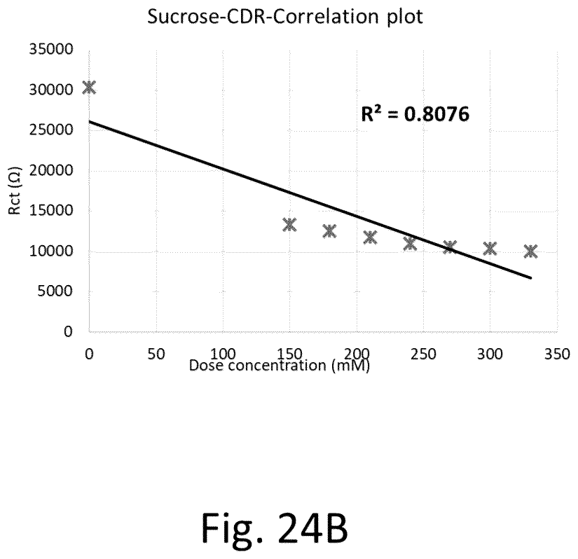

FIGS. 24A-24B show the Sucrose CDR/EIS measurements for Day 16. FIG. 24A shows the Nyquist plot, showing the imaginary (Z″) versus the real impedance (Z′). FIG. 24B shows the scatter plot of Rct versus sucrose dose, wherein a straight line is fitted through the scattered Rct values to get the CDR correlation plot.

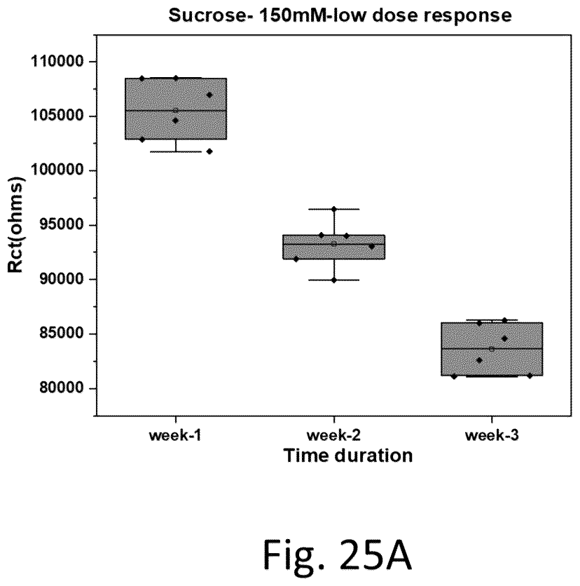

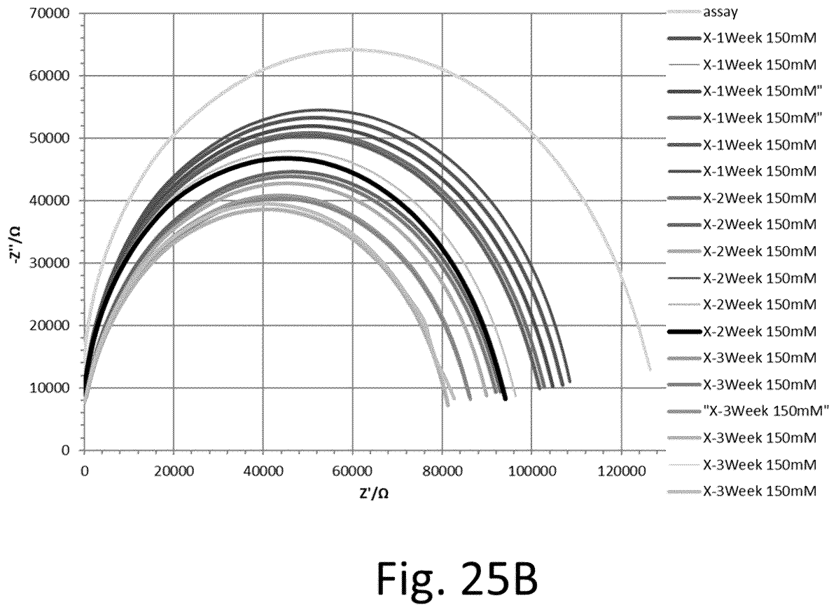

FIGS. 25A-25B show dose response curves indicating sensor surface layer stability from signal range consistency during weekly measurements over 3 weeks for a low-dose sample of sucrose (150 mM). FIG. 25A shows a dose-response curve generated by plotting Rct (charge transfer resistance, in Ohms) against time. FIG. 25B shows Nyquist curves with plots of imaginary part of impedance (Z″) versus the real part of the impedance (Z′).

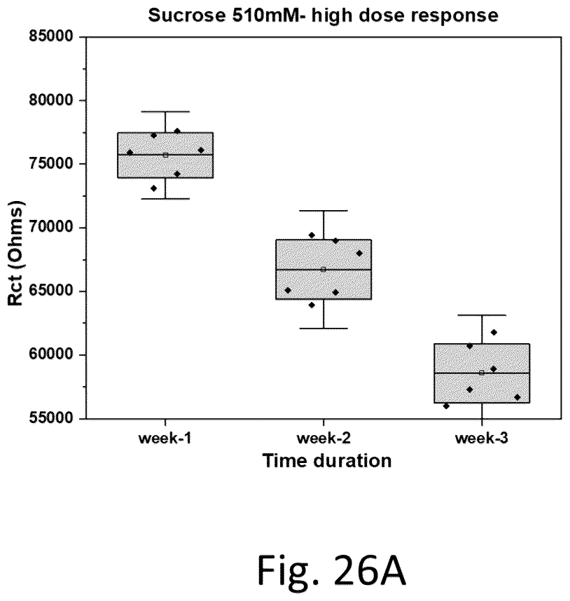

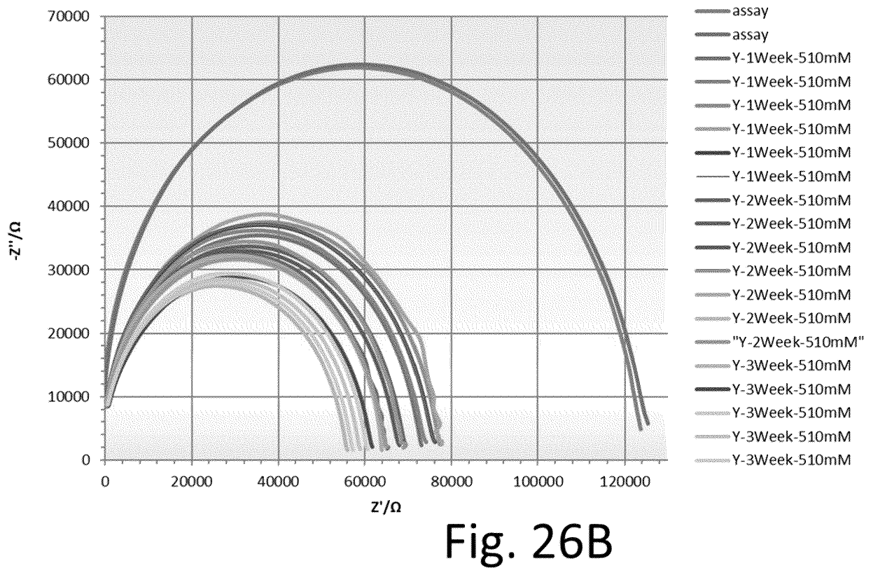

FIGS. 26A-26B show dose response curves indicating sensor surface layer stability from signal range consistency during weekly measurements over 3 weeks for a high-dose sample of sucrose (510 mM). FIG. 26A shows a dose-response curve generated by plotting Rct (charge transfer resistance, in Ohms) against time. FIG. 26B shows Nyquist curves with plots of imaginary part of impedance (Z″) versus the real part of the impedance (Z′).

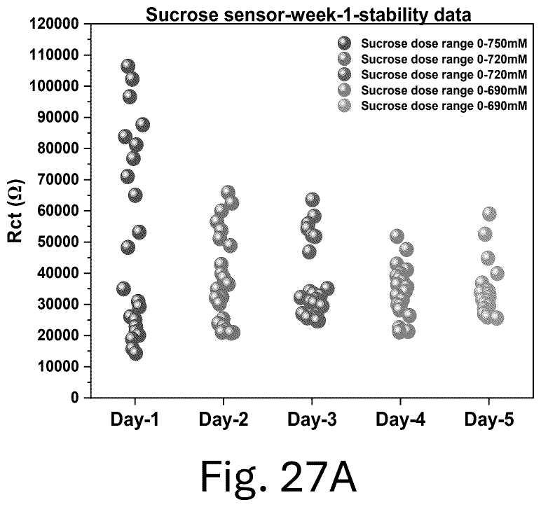

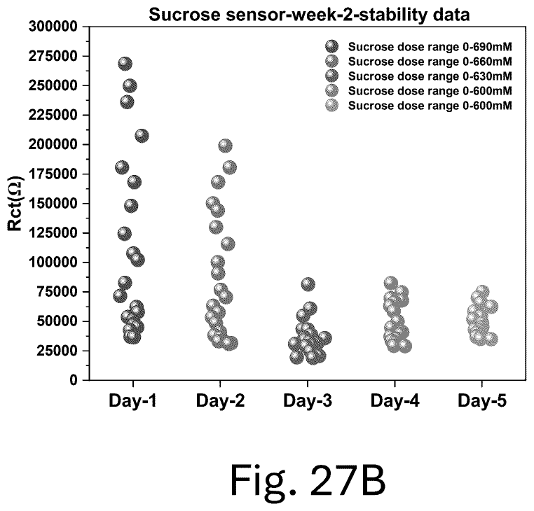

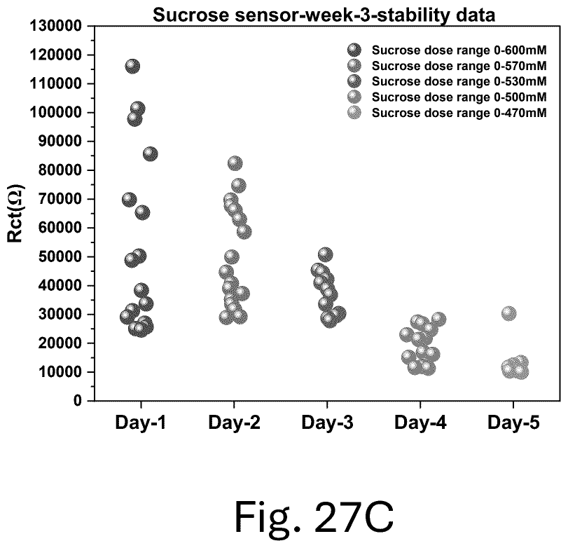

FIGS. 27A-27C show stability repeatability data at week 1 (FIG. 27A), week 2 (FIG. 27B) and week 3 (FIG. 27C) at sucrose dose ranges between 0 and 750 mM.

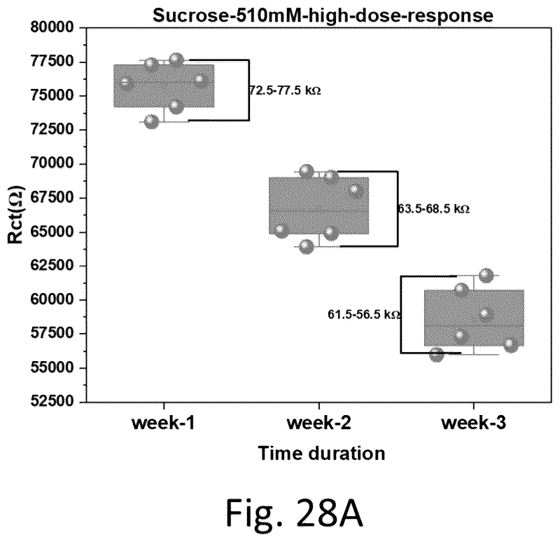

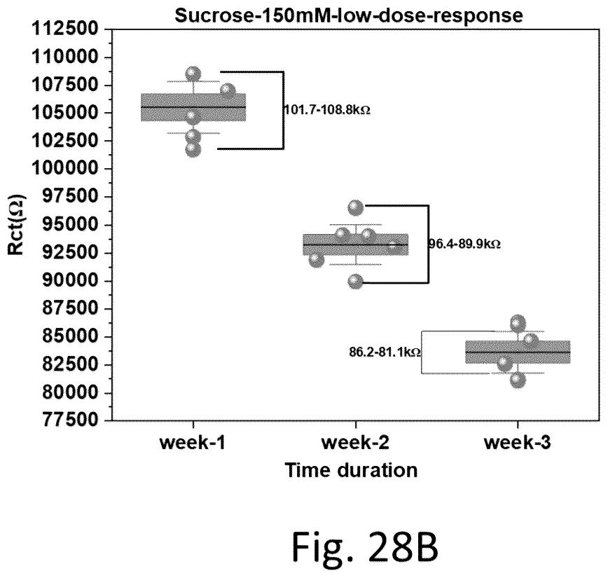

FIGS. 28A-28B show stability reproducibility data for weeks 1, 2, and 3 at sucrose high dose (510 mM, FIG. 28A) and sucrose low dose (150 mM, FIG. 28B).

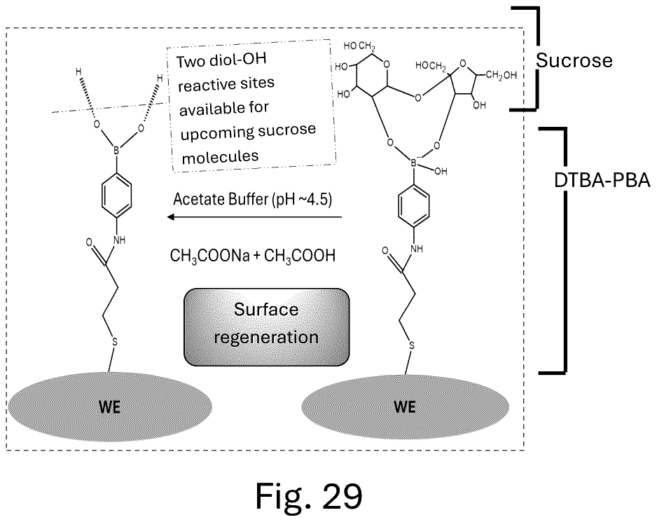

FIG. 29 shows the surface regeneration chemistry employed in the assay whereby the sucrose molecule is washed out of the assay, leaving unbound DTBA-PBA.

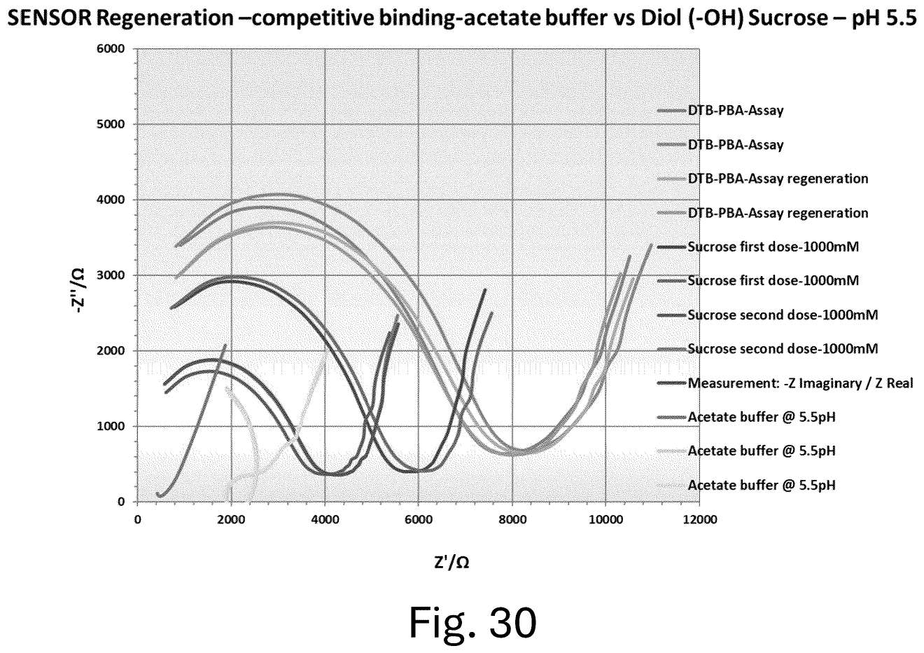

FIG. 30 shows competitive binding data from regeneration assay studies evaluating the acetate buffer pH. The competitive binding of acetate buffer vs diol was interrogated wherein the DTB-PBA-Assay concentration was about 5 mM, the acetate buffer concentration was about 10 mM, and the pH was about 5.5.

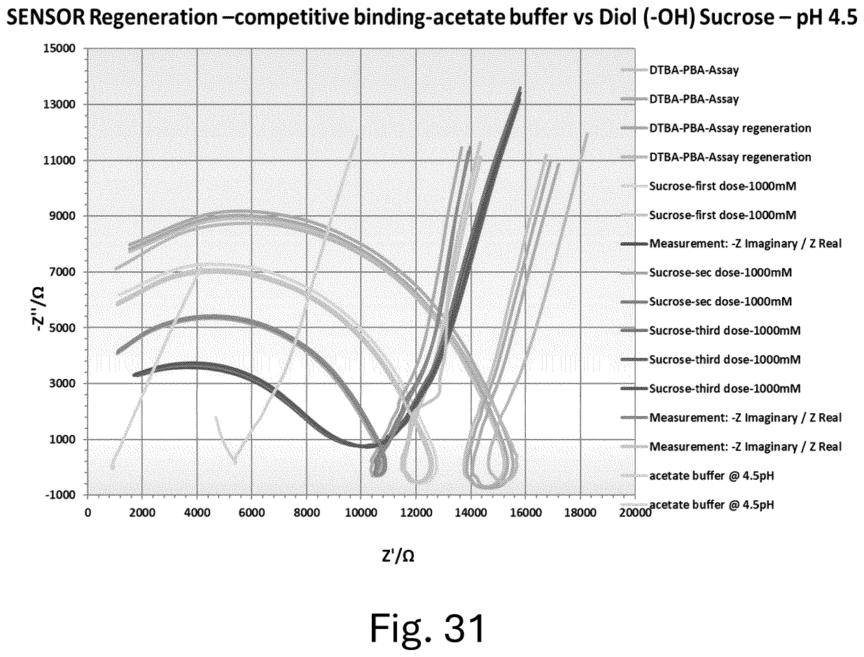

FIG. 31 shows competitive binding data from regeneration assay studies evaluating the acetate buffer pH. The competitive binding of acetate buffer vs diol was interrogated wherein the DTB-PBA-Assay concentration was about 10 mM, the acetate buffer concentration was about 10 mM, and the pH was about 4.5.

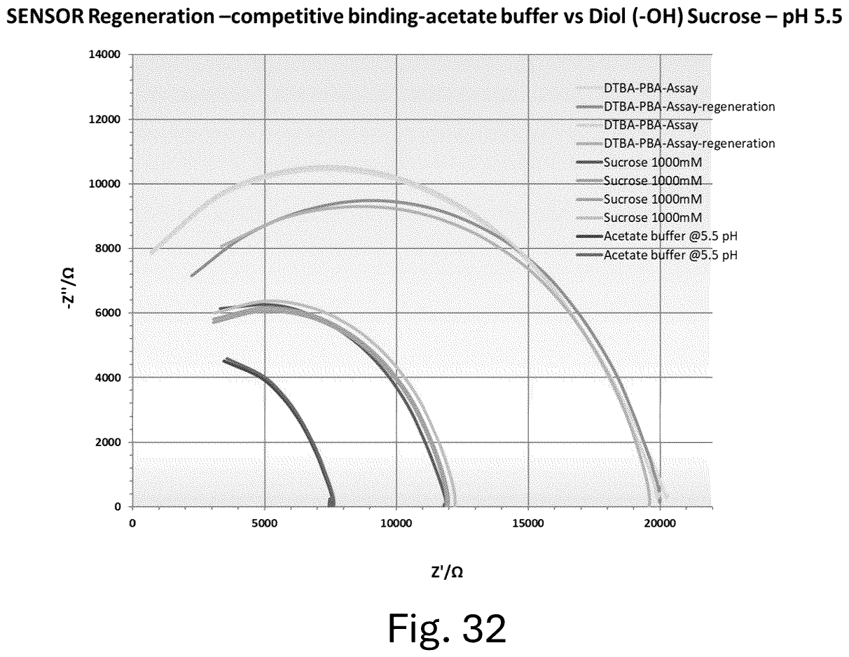

FIG. 32 shows competitive binding data from regeneration assay studies evaluating the acetate buffer pH. The competitive binding of acetate buffer vs diol was interrogated wherein the DTB-PBA-Assay concentration was about 10 mM, the acetate buffer concentration was about 10 mM, and the pH was about 5.5.

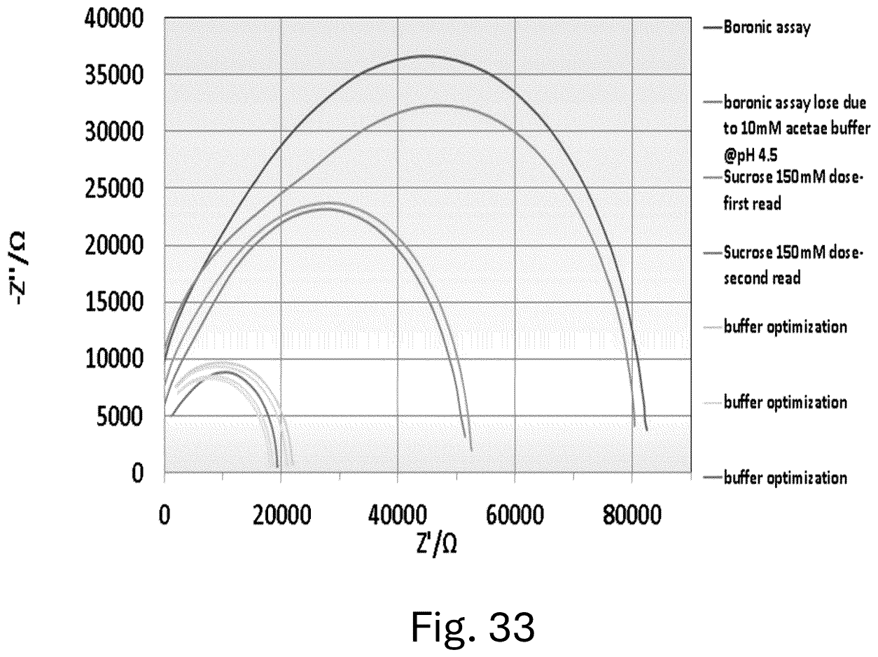

FIG. 33 shows the results of a buffer concentration with low concentrations of sucrose (between 1 mM and 150 mM) wherein the buffer pH was held constant (DTBA-PBA assay pH of 4.8, acetate buffer pH of 4.5).

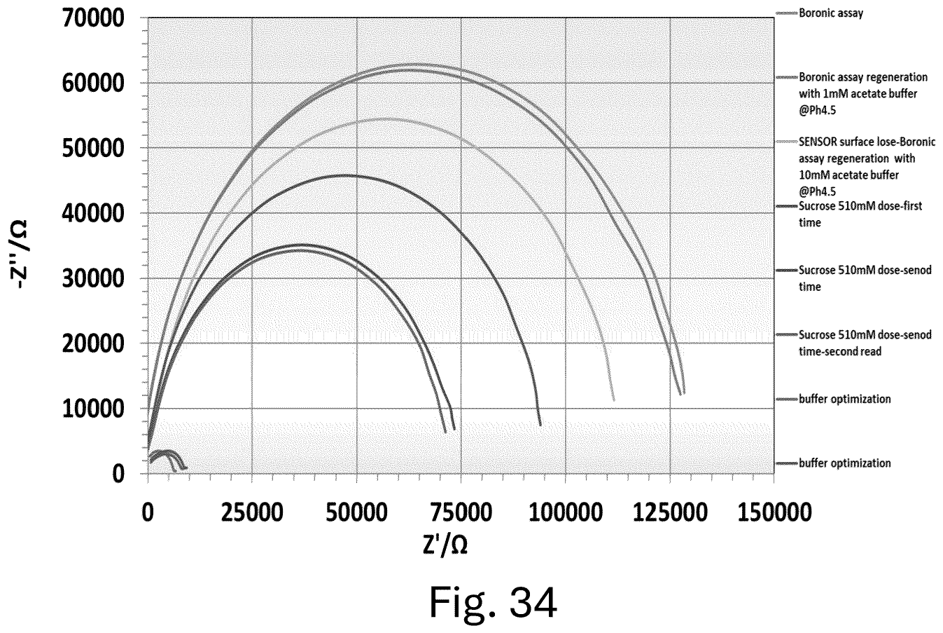

FIG. 34 shows the results of a buffer concentration with medium concentrations of sucrose (about 510 mM) wherein the buffer pH was held constant (DTBA-PBA assay pH of 4.8, acetate buffer pH of 4.5).

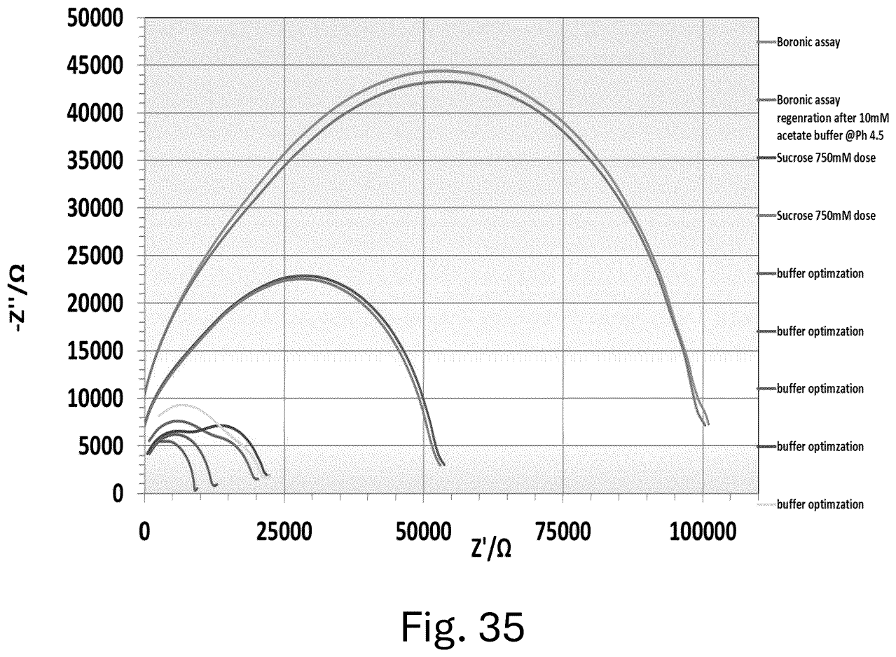

FIG. 35 shows the results of a buffer concentration with high concentrations of sucrose (about 750 mM) wherein the buffer pH was held constant (DTBA-PBA assay pH of 4.8, acetate buffer pH of 4.5).

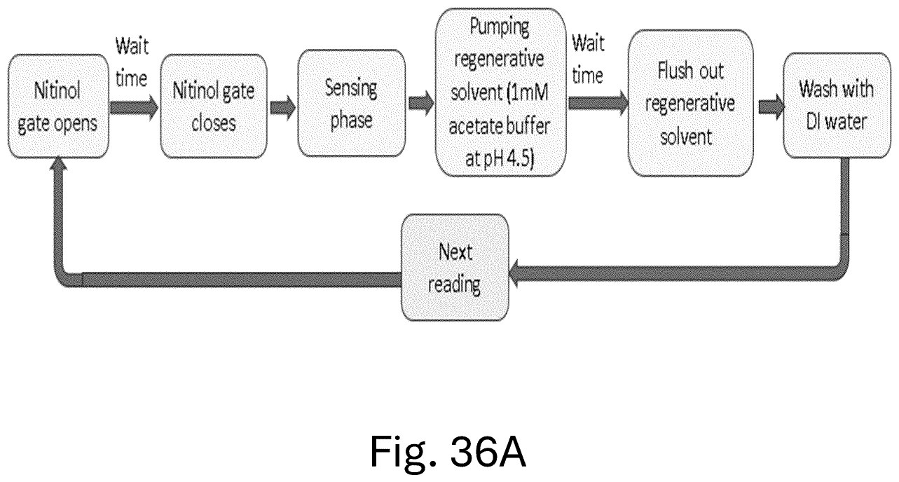

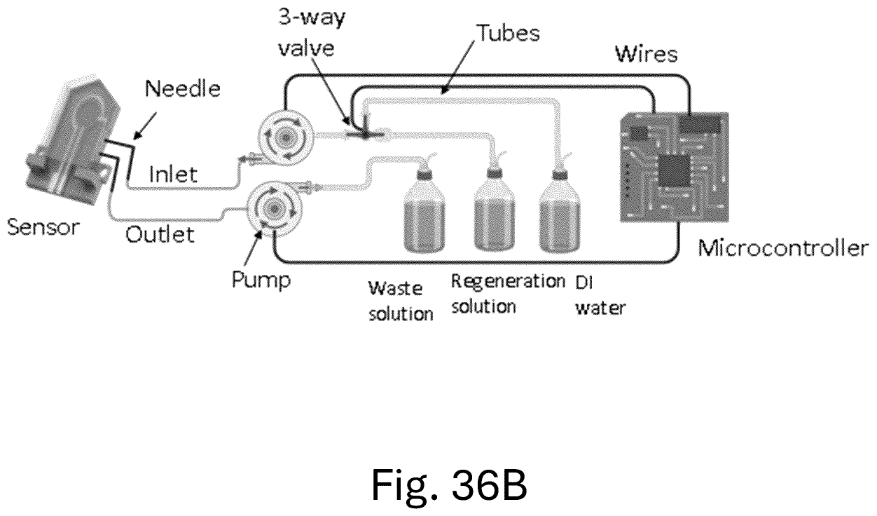

FIGS. 36A-36B show an overview of the sensing and regeneration washing processes. FIG. 36A depicts a flowchart of the sensing (steps 1-3) and regeneration (steps 4-6) processes. FIG. 36B shows a schematic illustration of the components of the regeneration washing process.

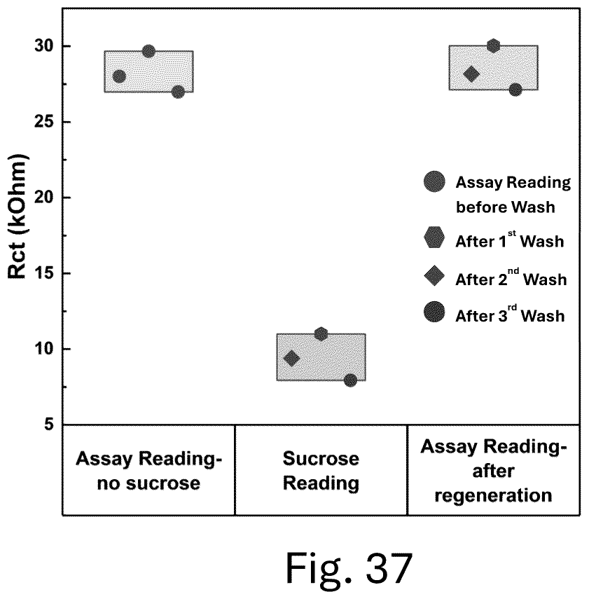

FIG. 37 shows results of the regeneration field test for showing Rct in kOhm as an assay reading with no sucrose (three readings before wash), a sucrose reading (after 1^{st}, 2^{nd}, and 3^{rd} wash), and an assay reading after regeneration (1^{st}, 2^{nd}, and 3^{rd} wash).

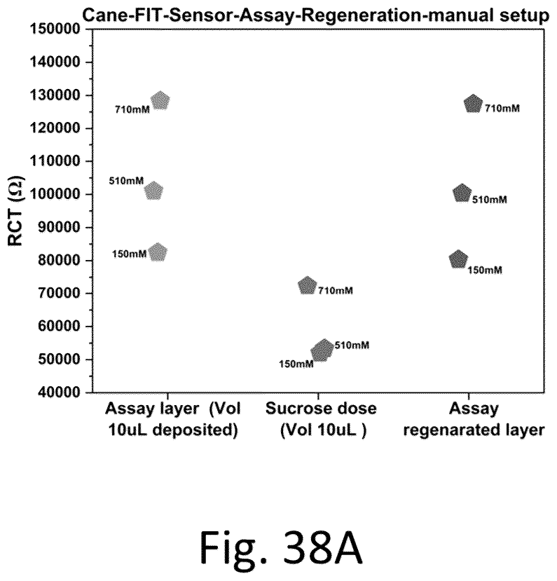

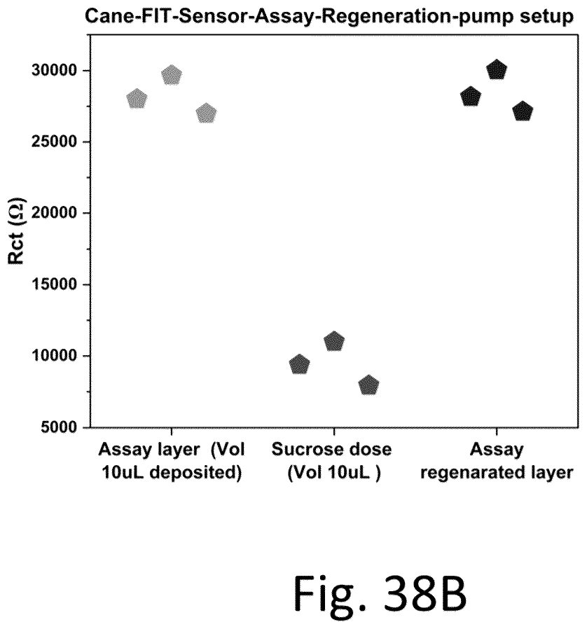

FIGS. 38A-38B show the comparative analysis, conducted in triplicate, for regeneration data of a sensor with a manual wash (no nitinol SMA wire gate or pump, FIG. 38A) and with the complete pump setup (FIG. 38B).

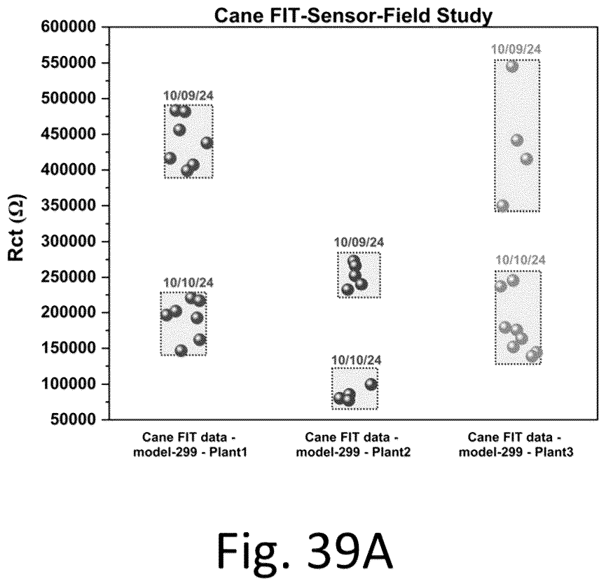

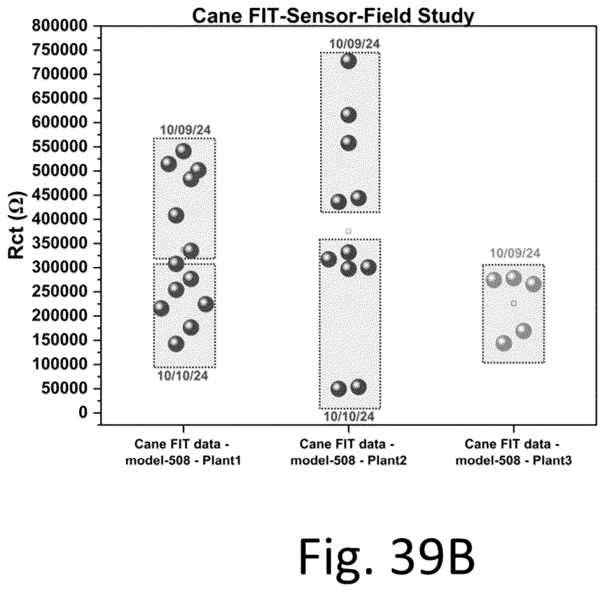

FIGS. 39A-39B show data collected from sensors implanted in live sugarcane plants. The model 299 cane plant (FIG. 39A) had a lower sucrose content than the model 508 plant (FIG. 39B).

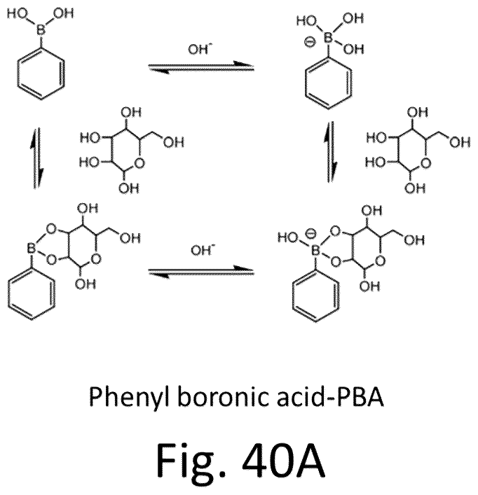

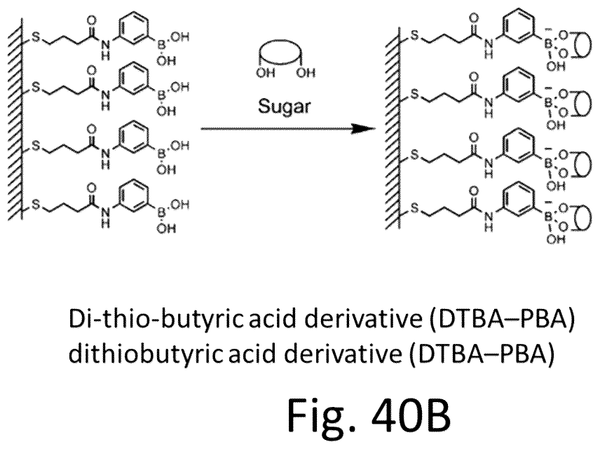

FIGS. 40A-40B show chemical reaction diagrams of the assay layer of the sensor. FIG. 40A shows the phenyl boronic acid (PBA). FIG. 40B shows the di-thio-butyric acid derivative (DTBA-PBA).

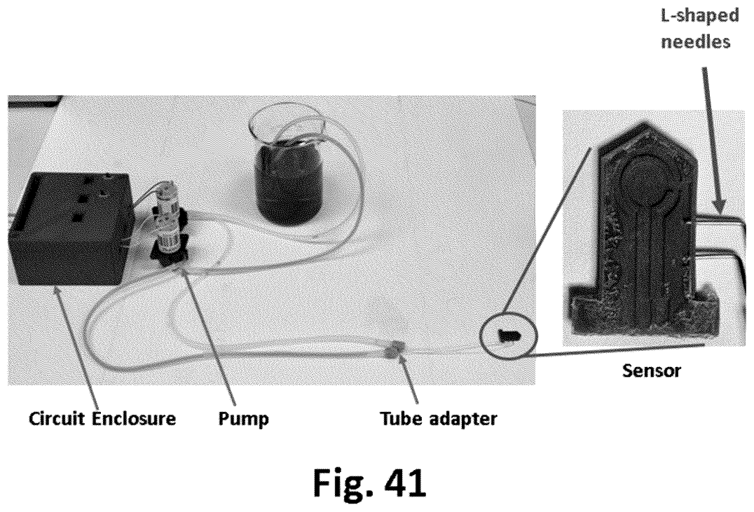

FIG. 41 shows an embodiment of regeneration system including a circuit enclosure, pump, tube adaptor, and sensor, including the L-shaped needles which allow for fluid ingress and egress into the sensor.

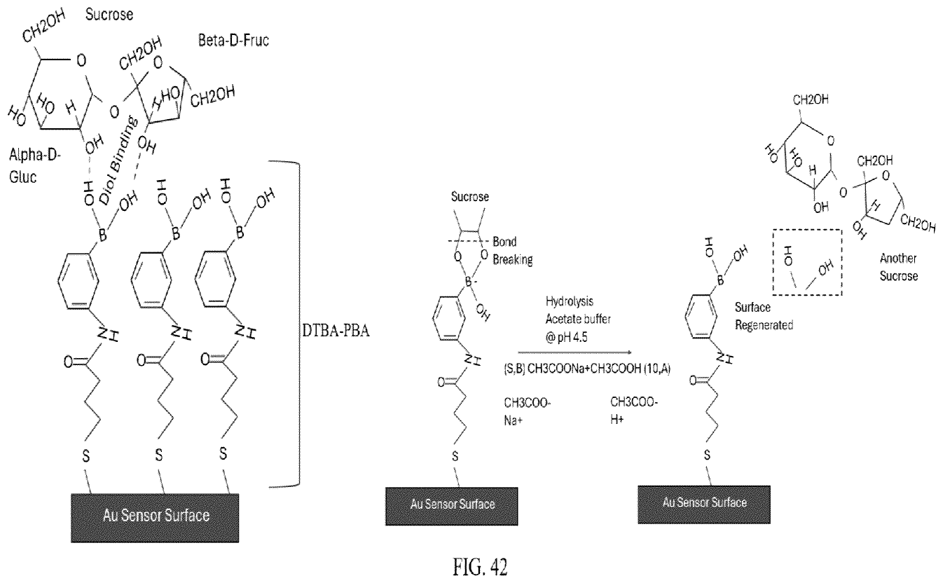

FIG. 42 shows a chemical reaction diagram of the competitive binding assay as part of the regeneration system under an embodiment comprising an acetate buffer at an acidic pH between 4.5 and 5.5.

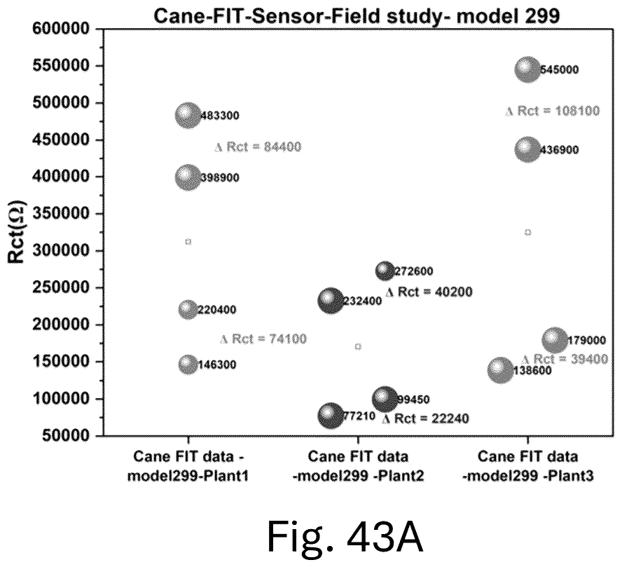

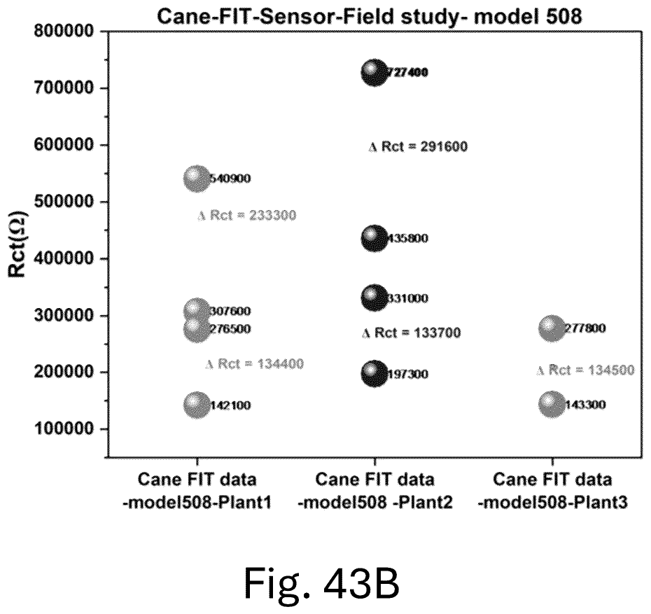

FIGS. 43A-43B show data collected from sensors implanted in live sugarcane plants. The model 299 cane plant (FIG. 43A) had a lower sucrose content than the model 508 plant (FIG. 43B). Data was collected in triplicate.

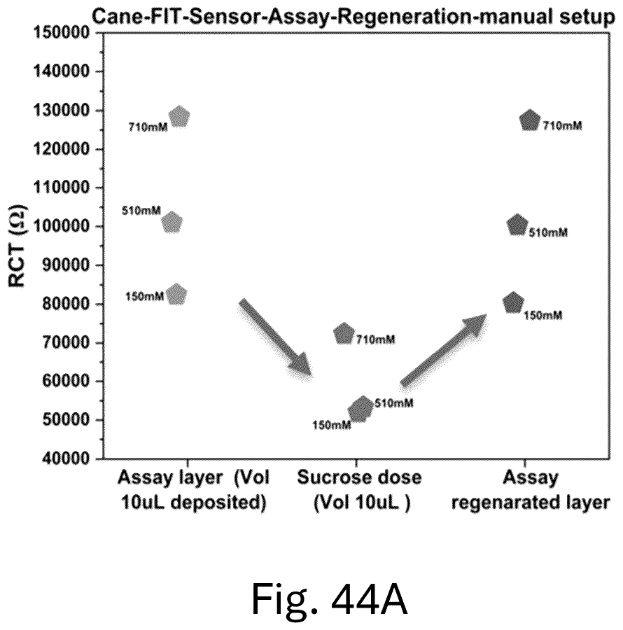

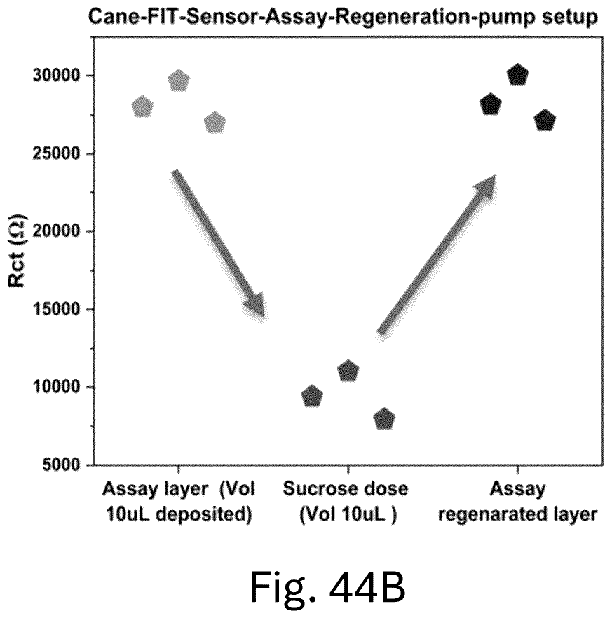

FIGS. 44A-44B show data collected from sensors implanted in live sugarcane plants demonstrating the sensor regeneration. As shown in FIG. 44A, known concentrations of sucrose solutions were used (1 replicate, 3 different concentrations to cover the entire sucrose range. As shown in FIG. 44B, cane juice was used (3 replicates, one Brix value of 18.3).

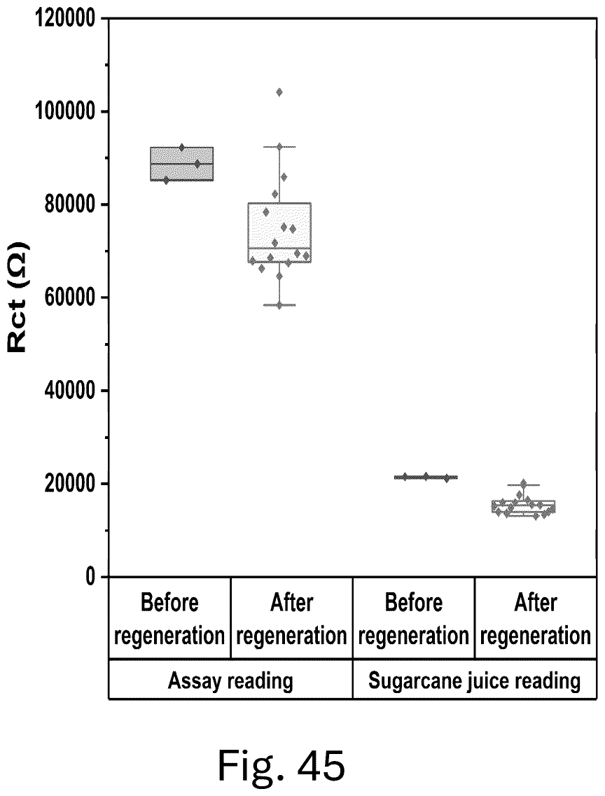

FIG. 45 shows data collected from sensors implanted in live sugarcane plants demonstrating the sensor regeneration.

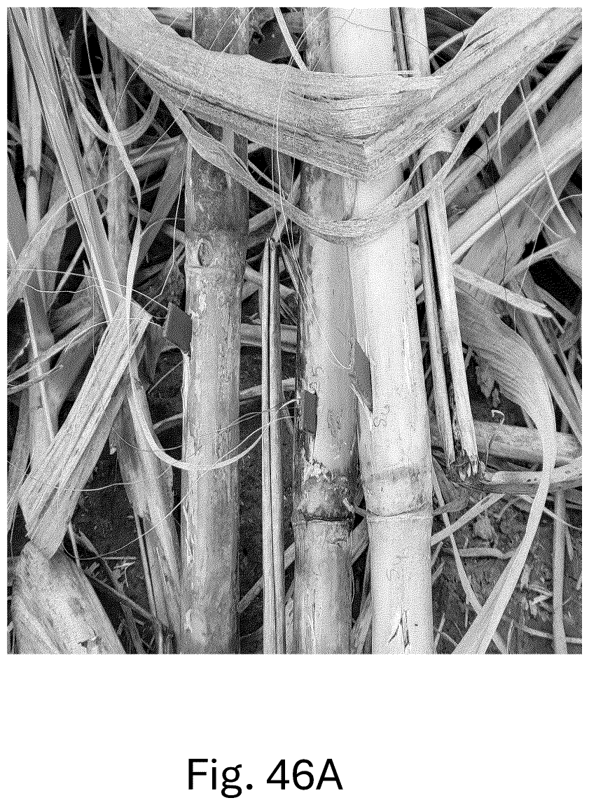

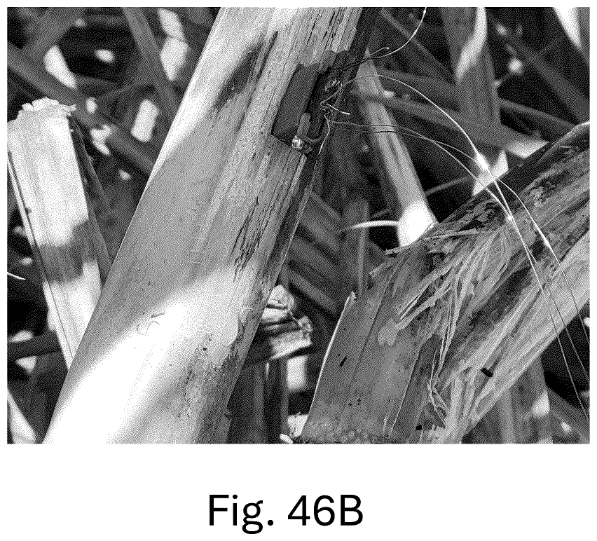

FIGS. 46A-46B show sensor installation in a sugarcane field in Houma, Louisiana.

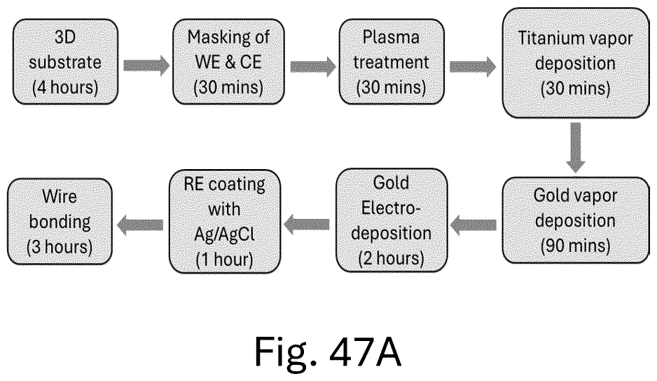

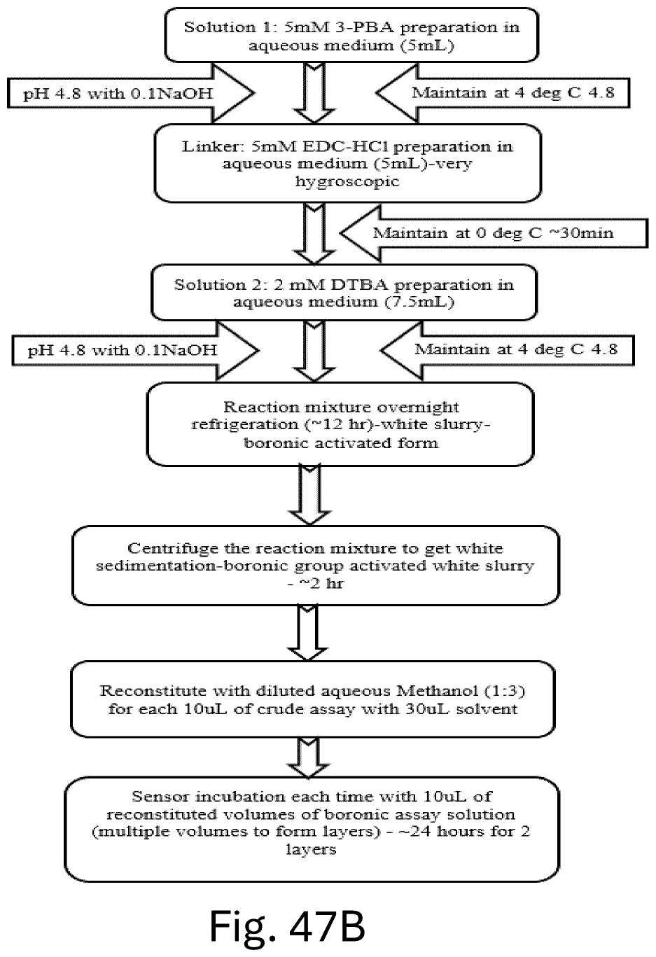

FIG. 47A shows a flow diagram of the sensor electrode fabrication process. FIG. 47B shows a flow diagram for boronic assay preparation and incubation.

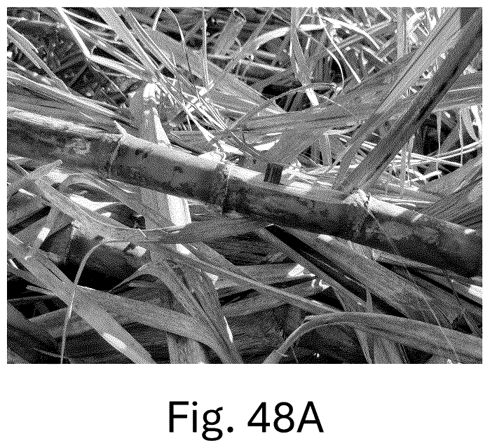

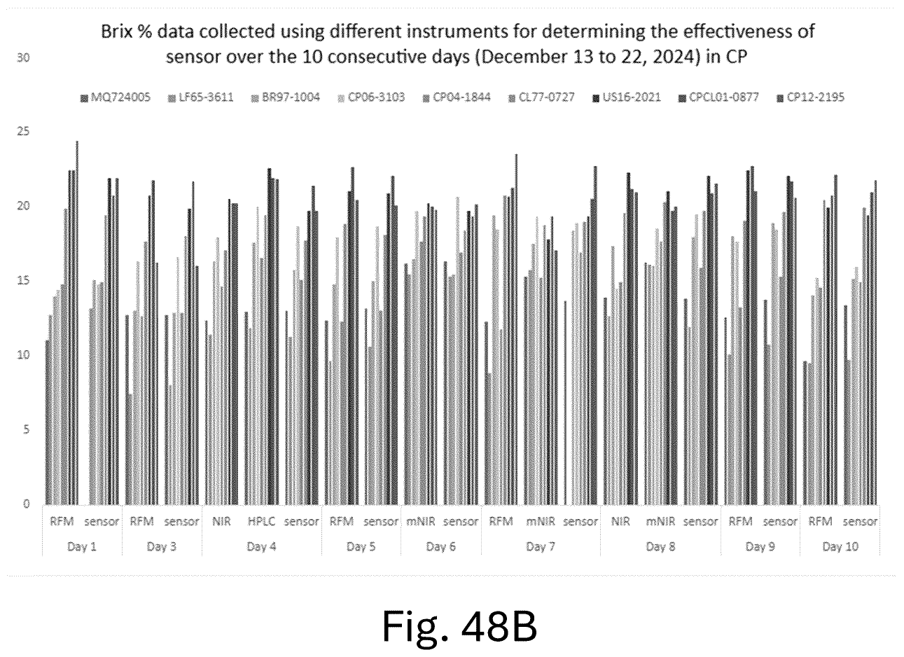

FIG. 48A is an optical image of sensors installed in the cane stalks in ARS field station at Canal Point, FL. FIG. 48B shows Brix % data collected using different instruments for determining the effectiveness of sensors over 10 consecutive days.

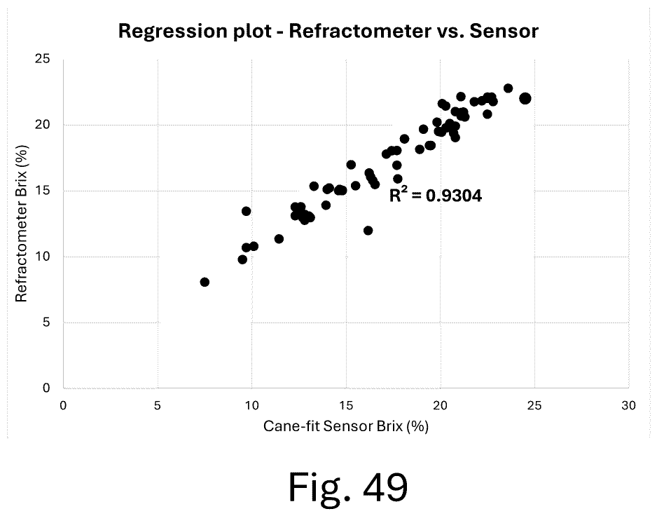

FIG. 49 is a regression plot comparing Brix measures by refractometer vs. sensors.

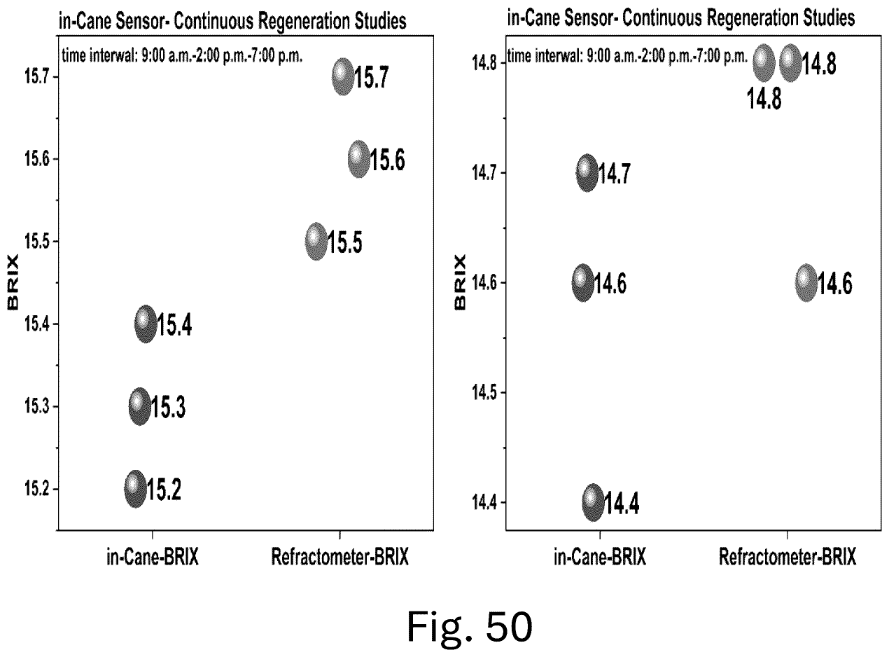

FIG. 50 shows in-cane sensor regeneration demonstrated at 3 time points in a full day with 2 different cane models and Brox values compared with standard refractometer readings.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

### Definitions

A “plant sensor” means a device configured to attach to a plant and to sense one or more conditions, including, but not limited to, pH, temperature, water content, or humidity and/or one or more analytes, including, but not limited to phytohormones, phytochemicals, or volatile organic compounds. In a preferred embodiment, the analyte is sucrose. In some embodiments, the plant sensor may be a plant patch or a leaf patch, such as a microneedle patch, configured to attach to a part of a plant, such as a plant stem or one or more leaves. In some embodiments, the plant sensor is flexible.

A “plant sensor system” means one or more components in addition to the plant sensor, including, but not limited to, a potentiostat, one or more of the processors, a communication unit, a power supply unit, and/or a data acquisition system. A plant sensor system may include additional components to perform any of the techniques described herein.

A “processor” means one or more microprocessors, central processing units (CPUs), processing circuitry, computing devices, one or more microcontrollers, digital signal processors, or like devices or any combination thereof, regardless of the architecture (e.g., chip-level multiprocessing/multi-core, RISC, CISC, Microprocessor without Interlocked Pipeline Stages, pipelining configuration, simultaneous multithreading). In some embodiments, the processor is operatively connected to memory. The processor and memory may be connected externally or internally.

“Bioagent” means a biological substance, including, but not limited to peptides, enzymes, polypeptides and proteins, nucleotides/nucleic acid, or polynucleotides, any organism, cell, or virus, living or dead, or a nucleic acid derived from such an organism, cell or virus.

“Non-invasive” means plant tissue is minimally affected by any sensors or measurements. In some embodiments, a non-invasive sensor is attached to the plant without any damage, for example without penetrating the tissue of the plant, including the leaf, stem, or roots. In some embodiments, a non-invasive sensor is attached to the plant with minimal damage. In some embodiments, a non-invasive sensor is configured to penetrate plant tissue by a few microns in depth. In some embodiments, a non-invasive sensor is configured to penetrate plant tissue by 1-100 micrometers, or a non-invasive sensor is configured to penetrate plant tissue by less than 90, 80, 70, 60, 50, 40, 30, 20, 10, 9, 8, 7, 6, 5, 4, 3, 2, or 1 micrometers.

“Biocompatible polymer” means any synthetic (man made) or natural polymers which are suitable to be used in the close vicinity of a living system or work in intimacy with living tissue. Examples of biocompatible polymers include, but are not limited to, polyethylenes, polyvinyl chlorides, polyamides, such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers, such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates, such as ethylene vinyl acetate copolymer, homopolymers and copolymers of acrylates, such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers, such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

“Computer-readable medium” means any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include random access memory (RAM) or dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, SecureDigital (SD™) memory card, USB Flash Drives, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

As used herein, the term “real-time monitoring system” a means a device comprising one or more components in addition to the sensor or sensor array, including, but not limited to, connectors, joining parts, a wireless transmitter, and/or a data acquisition system. A real-time monitoring system may include additional components to perform any of the techniques described herein.

As used herein, the terms “approximately” and “about,” as applied to one or more values of interest, refer to a value that is +/−10% of the recited value.

### Regenerative Plant Sensor

Provided herein is a regenerative plant sensor, comprising a flexible substrate embedded with one or more shape memory alloy (SMA) wires, a substrate comprising a sensor layer, the sensor layer comprising a working electrode (WE), a counter electrode (CE), and a reference electrode (RE), wherein one or more electrodes on said sensor layer are functionalized with one or more of a first metal, a second metal, and a composition comprising a synthetic polymer and a hydrogel, a microfluidic system in communication with a fluid chamber and the sensor layer, one or more pumps in communication with the microfluidic system, a microcontroller in electrical communication with the sensor layer, the one or more pumps, and the one or more shape memory alloy (SMA) wires. In some aspects, the one or more SMA wires form a fluid gate in the flexible substrate. In some aspects, the fluid chamber is filled with a regeneration solution.

In some aspects, the WE and CE are functionalized with a sputter-coated layer of the first metal. In some aspects, the WE is further functionalized with an electrically deposited layer of the first metal. In some aspects, the first metal is gold. In some aspects, the RE is functionalized with a layer of the second metal. In some aspects, the second metal is silver and/or silver chloride. In some aspects, the WE is further functionalized with an assay layer.

FIG. 1 shows a non-limiting specific embodiment of the regenerative plant sensor 50. The sensor includes a substrate comprising a sensor layer 100. In some embodiments, the substrate comprising a sensor layer has the dimensions of about 2.5 cm by about 1 cm. In some aspects, the substrate comprising a sensor layer 100 is three-dimensional (3D) printed. In some aspects, the substrate comprising a sensor layer 100 is chemically functionalized as described herein to form a sucrose sensor which can be easily inserted into the plant stalk with minimal damage. Fabrication begins with a substrate comprising a sensor layer 100 further comprising a working electrode (WE), a counter electrode (CE), and a reference electrode (RE) 130. In some aspects, the substrate comprising the sensor layer is 3D printed with a stereolithography (SLA) printer. In some aspects, the substrate comprising the sensor layer is plasma treated to increase hydrophilicity. In some aspects, a WE and a CE masking is patterned using a vinyl cutter. In some aspects, a titanium adhesion layer is deposited onto the sensor layer. In some aspects, the titanium adhesion layer is 1 to 100 nm thick. In some aspects, the titanium adhesion layer is about 10 nm thick. In some aspects, gold is sputter-coated onto the WE and CE of the substrate to create a sputter-coated gold layer 110 and 120. In some aspects, the sputter-coated gold layer is 100 to 500 nm thick. In some aspects, the sputter-coated gold layer is about 200 nm thick. In some aspects, gold is electrodeposited to form a gold electrodeposited layer 130 on the WE and CE in order to improve conductivity and increase the surface area for assay immobilization. In some aspects, the electrodeposition is performed using cyclic voltammetry in a gold (III) chloride trihydrate solution. In some aspects, silver and silver chloride coating are applied to a reference electrode surface of the sensor to form a silver and silver chloride layer 140. In some aspects, the electrodeposited gold layer 130 is immobilized with a composition comprising a synthetic polymer and a hydrogel, preferably a polymer di-thio-butyric acid (DTBA) derivative, which selectively binds to sucrose, forming an assay layer 150 on the sensor. In some aspects, the assay layer is formed from a boronic assay solution. In some aspects, the boronic assay solution is produced by a method comprising: a) preparing a first solution comprising 3-aminophenyl boronic acid hemi sulfate salt (3-PBA) at a pH of 4-6, b) preparing a linker solution comprising 1-ethyl-3-(3-dimethylaminopropyl) carbodiimide hydrochloride (EDC-HCl), c) preparing a second solution comprising 4,4′-dithio-3-di(n-butyric acid) (DTBA) at a pH of 4-6, d) mixing the first solution with the linker solution to form a first mixture, c) adding the first mixture to the second solution to form a reaction mixture, f) incubating the reaction mixture at 4° C. for about 8-16 hours, then centrifuging to sediment an assay slurry, and g) isolating the assay slurry, then reconstituting the assay slurry in a methanol:water solution to form the boronic assay solution. In some aspects, the first solution comprises 1-10 mM 3-PBA, or about 5 mM 3-PBA. In some aspects, the first solution has a pH of about 4.8. In some aspects, the first solution is maintained at about 4° C. In some aspects, the linker solution comprises 1-10 mM EDC-HCl, or about 5 mM EDC-HCl. In some aspects, step b) further comprises incubating the linker solution at about 0° C. for about 30 minutes. In some aspects, the second solution comprises 1-5 mM DTBA, or about 2 mM DTBA. In some aspects, the second solution has a pH of about 4.8. In some aspects, the second solution is maintained at about 4° C. In some aspects, step c) further comprises stirring the reaction mixture for 1-3 hours, or about 2 hours. In some aspects, step f) comprises centrifuging the reaction mixture at 2000-5000 rpm, or about 3500 rpm. In some aspects, step f) comprises centrifuging the reaction mixture for 1-3 hours, or about 2 hours. In some aspects, the methanol:water solution has a v:v ratio of 1:3. Collectively, the fabricated sensor including the sputter-coated gold layer, electrodeposited layer, silver and silver chloride coatings, and assay layers are also referred to herein as the “sensor layer.”

In one aspect, the disclosure provides a regenerative plant sensor shown in FIGS. 3-6, comprising a flexible substrate embedded with one or more shape memory alloy (SMA) wires to form a fluid gate in the flexible substrate (“SMA wire gate”) 200, a sensor layer comprising a metal and a composition comprising a synthetic polymer and a hydrogel, as described above, a microfluidic system in communication with a fluid chamber or channel 210 and the sensor layer, one or more pumps in communication with the microfluidic system, a microcontroller in electrical communication with the sensor layer, the one or more pumps, and the SMA wire gate 200, and wherein the fluid chamber is filled with a regeneration solution 320. In some embodiment, the microfluidic system comprises an inlet 220 and an outlet 230. In some embodiments, the SMA wire gate 200 is a 1-way actuation gate. In some embodiments, the SMA wire gate 200 is a 2-way actuation gate.

In some embodiments, the sensor layer may be functionalized with a sputter-coated metal layer 110. In some embodiments, the sensor layer may be functionalized with an electrically deposited metal layer 120. In some embodiments, the metal may be gold.

In some embodiments, the synthetic polymer may interact with a sugar or a hormone. In some embodiments, the synthetic polymer binds to a sugar or a hormone. In certain aspects, the synthetic polymer forms one or more hydrogen bonds with the sugar or the hormone. In some aspects, the polymer may include functional groups such as hydroxyl, amine, or carboxyl groups which further support hydrogen bonding or other intermolecular interactions which may stabilize the bond between the polymer and the sugar or the hormone. In some embodiments, the polymer forms one or more ionic bonds, covalent bonds, or hydrophobic interactions with the sugar or the hormone. In some embodiments, the polymer may be di-thio-butyric acid (DTBA) or a derivative thereof. In some embodiments, the sugar may be sucrose.

In some embodiments, the hydrogel may be a polyvinyl alcohol (PVA)-based hydrogel.

In some embodiments, the SMA wire gate 200 may be closed in a first state. In some embodiments, the fluid gate may be open in a second state.

In some embodiments, the one or more SMA wires comprising the SMA wire gate may comprise nickel and titanium. In some embodiments, the one or more SMA wires may comprise at least one one-way actuated wire and at least one two-way actuated wire.

In some aspects, and as shown in FIG. 8, the regeneration system includes a circuit enclosure 300, a pump enclosure 310, a regeneration solution 320, a tube adaptor 330, and the regenerative plant sensor 50. In some embodiments, the circuit enclosure includes a first switch 400, a second switch 405, a microcontroller 410, a motor controller circuit 420. In some embodiments, the elements of the circuit enclosure are connected to a first pump 430, a second pump 435, one or more tube adaptors 335, and the regenerative plant sensor 50, as described in FIG. 7.

In some embodiments, the regeneration solution may comprise an acetate buffer. In some embodiments, the regeneration solution may comprise an acetate buffer at a concentration of about 1.0 mM, about 2.0 mM, about 5.0 mM, or about 10.0 mM, or between about 0.5 mM and about 1.0 mM, between about 2.5 mM and about 7.5 mM, between about 1.0 mM and about 10.0 mM, or between about 5.0 mM and about 15.0 mM. In some embodiments, the regeneration solution may have a pH of about 4, about 4.5, about 5, about 5.5, about 6, or about 6.5, or between about 4 and 4.5, between about 5 and about 5.5, between about 4 and about 5, between about 4.5 and about 5.5, or between about 5 and about 6.

In some embodiments, the flexible substrate may be a thermoplastic and/or thermosetting resin. In some embodiments, the thermoplastic and/or thermosetting resin may be polydimethylsiloxane, polybutylene adipate terephthalate (PBAT, Ecoflex™) or a silicone rubber. In some embodiments, the flexible substrate may have a thickness of 10 μm to about 50 μm, about 50 μm to about 500 μm, about 100 μm to about 400 μm, about 50 μm to about 1000 μm, about 100 μm to about 1500 μm, about 50 μm to about 2000 μm, or about 1000 μm to about 3000 μm.

In some embodiments, the sensor layer is bioagent-free.

In some embodiments, the regenerative plant sensor further may further comprise a data acquisition system, wherein the data acquisition system comprises a processor; a communication unit; a memory unit; and wherein the data acquisition system is in communication with the sensor layer.

In some embodiments, the memory unit may be communicatively coupled to the processor, the memory unit having stored thereon computer software comprising a set of instructions that, when executed by the processor, causes the data acquisition unit to receive sensor data from the one or more sensors; and send, via the communication unit, the sensor data to an external device. In some embodiments, the memory unit may comprise a non-transitory computer-readable medium. In some embodiments, the sensor data may be sent, via the communication unit, to an Internet of Things (IoT) cloud server configured to interact with one or more IoT-capable devices.

In some embodiments, the regenerative plant sensor may further comprise a power supply unit. In some embodiments, the microcontroller and the data acquisition system may each comprise a power supply unit.

In some embodiments, the instructions may comprise a signal calibration.

In some embodiments, the regenerative plant sensor may have a total volume of about 200 to about 1,000 cm³, about 300 to about 800 cm³, or about 400 to about 600 cm³.

In some aspects, the total power consumption for the cycle may be about 1 W to about 5 W, about 1 W to about 10 W, about 1 W to about 15 W, about 2 W to about 8 W, or about 2 W to about 15 W. In some aspects, the power supply unit may supply power for about 1 cycle to about 5 cycles, about 5 cycles to about 100 cycles, about 10 cycles to about 75 cycles, about 20 cycles to about 60 cycles, about 25 cycles to about 75 cycles, or about 30 cycles to about 50 cycles.

In some aspects, the analyte may be measured about every 1 minute to about 5 minutes, about 3 minutes to about 25 minutes, about 5 minutes to about 15 minutes, about 7 minutes to about 10 minutes, or about 10 minutes to about 20 minutes.

### Electromechanical and Chemical Regeneration

The plant sensor is regenerative, significantly extending its shelf life to several months. This dual-mode regeneration approach integrates both electromechanical and chemical processes. The electromechanical method involves placing an electrically actuated SMA wire gate over the surface of the sensor. The chemical process uses built-in microfluidics to direct a regeneration solution across the surface of the sensor.

Electromechanical regeneration of the sensor is achieved, as described herein, using an SMA wire gate as shown in FIGS. 4-6. Two types of nitinol wires (two-way (FIG. 4) or two-way (FIG. 5) actuated) are embedded within a flexible and biocompatible polymer, such as polydimethylsiloxane (PDMS) or Ecoflex, to develop the SMA wire gate 200. The sensor's operation cycle begins by applying a potential to actuate the SMA wire gate, allowing sap to reach the sensor surface. The SMA wire gate may take a period of seconds to fully open. Once the sucrose-containing sap has covered the sensor surface for a set period of time, such as a few minutes, the SMA wire gate is closed by applying an opposite potential. The measured sucrose level is then displayed based on data collected by the sensor and compiled by a data logger.

Chemical regeneration of the sensor is achieved, as described herein, via a microfluidic system inside the sensor. A first pump and a second pump are connected to the fluid inlet and fluid outlet ports of the sensor, and said first pump and second pump are operated with a maximum of 5V power supply, and optionally include a speed control feature. Two L-shaped needles are attached to the inlet and outlet to enable solution pumping inside the microfluidic channel. Approximately 100-120 μL of solution is required for each washing step.

The first pump 430 is activated to channel the regeneration fluid through the microfluidic system for a period of minutes, wherein the regeneration solution is in contact with the sensor surface (see, e.g., FIG. 7). The regeneration fluid, which contains acetate buffer calibrated to a specific concentration and pH, breaks down the DTBA-sucrose bonds, freeing the two diol-OH reactive sites on the DTBA derivative to bind with new sucrose molecules. This chemical regeneration process takes a period of minutes to fully refresh the sensor surface.

Afterward, the second pump 435 is activated to flush out the fluid from the sensor surface. This process takes a period of minutes.

Both the first pump 430 and the second pump 435 are controlled by the same microprocessor that sends excitation signals to the sensor and the SMA wire gate, resulting in a compact system with a minimal footprint of about 12×6×7 cm³. This entire process is repeated in the next operation cycle of the sensor, taking a period of minutes. The entire regeneration process is fully automated by the onboard microprocessor and integrated fluidics system.

In some aspects, the step of applying the first electrical potential may open the SMA wire gate in about 10 to about 20 seconds, in about 10 to about 30 seconds, in about 10 to about 45 seconds, in about 10 to about 60 seconds, in about 15 to about 30 seconds, in about 15 to about 45 seconds, or in about 15 to about 75 seconds. In some aspects, the step of apply the second electrical potential may close the SMA wire gate in about 10 to about 20 seconds, in about 10 to about 30 seconds, in about 10 to about 45 seconds, in about 10 to about 60 seconds, in about 15 to about 30 seconds, in about 15 to about 45 seconds, or in about 15 to about 75 seconds.

In some aspects, the analyte may be measured for about 1 to about 2 minutes, about 1 to about 3 minutes, about 1 to about 4 minutes, about 1 to about 5 minutes, or about 2 to about 3 minutes, about 2 to about 4 minutes, about 2 to about 5 minutes, about 2 to about 6 minutes, or about 2 to about 7 minutes.

In some aspects, the data acquisition system may process the data from the sensor layer to produce a data output. In some aspects, the processed data of the data output are stored in the memory unit. In some aspects, the processed data may be sent, via the communication unit, to the IoT cloud server configured to interact with the one or more IoT-capable devices.

In some aspects, the regeneration solution may be in contact with the sensor surface for about 1 minute to about 5 minutes, about 1 minute to about 10 minutes, about 2 minutes to about 4 minutes, about 2 minutes to about 8 minutes, about 3 minutes to about 6 minutes, about 3 minutes to about 9 minutes, or about 4 minutes to about 5 minutes. In some aspects, the regeneration solution may be removed from the sensor layer in about 10 to about 60 seconds, about 10 to about 30 seconds, about 15 to about 20 seconds, or about 15 to about 45 seconds.

In some aspects, the measurement cycle may be completed in about 1 minute to about 10 minutes, about 2 minutes to about 20 minutes, about 3 minutes to about 12 minutes, or about 5 minutes to about 10 minutes.

In some aspects, the amount of analyte in the plant sap may be obtained from the IoT capable device. In some aspects, the analyte may be about 10 to about 100 mM, about 100 mM to about 500 mM, about 100 mM to about 1,000 mM, about 150 mM to about 750 mM, or about 500 to about 2,000 mM. In some aspects, the analyte may be about 1% to 10%, about 1% to about 25%, about 1% to about 40%, about 5% to about 25%, about 5% to about 35%, or about 5% to about 75%.

In some aspects, the signal corresponding to an amount of analyte in the plant sap may be measured at a resolution of about 1 mM to about 100 mM, about 1 mM to about 25 mM, about 5 mM to about 50 mM, about 5 mM to about 75 mM, or about 10 mM to about 40 mM. In some aspects, the signal corresponding to an amount of analyte in the plant sap may be measured at a resolution of about 0.015% to about 1.5%, about 0.05% to about 0.25%, about 0.1% to about 1%, or about 0.25% to about 0.75%.

### Data Collection and Output

The integrated sensor system can provide sucrose measurements in about minutes, with a total power consumption of about 7 W, eliminating the need for destructive and labor-intensive juice sampling from the plant. Additionally, all data is collected and processed automatically on-site, eliminating the need for any manual intervention.

To estimate the system's total power consumption, each of the first and second pumps uses around 1.82 W, the nitinol SMA wire gates use around 1.2 W during both the opening and closing of the gate, the microprocessor unit consumes roughly 0.4 W, and other electronics in the data logger (not shown) draw approximately 0.5 W. This results in a total power consumption of around 7 W for a complete sensing cycle.

In one embodiment, the entire operation of sensing and regenerating lasts about 10 minutes, so that a 5V battery with a 10,000 mAh (or 10 Ah) rating can power the system for approximately 43 cycles. All the electronics and mechanical components of the system are reusable, with only the battery requiring periodic replacement.

Analyte detection sensitivity may be a change in the sensor/instrument's signal per unit change in analyte concentration, indicating how responsive the device is to the presence of low analyte levels. In some embodiments, analyte detection sensitivity may be a percentage of a signal corresponding to an amount of analyte after a measurement cycle compared to a signal corresponding to the amount of analyte prior to completing a measurement cycle.

### In Situ Use

Provided herein is a method of measuring an analyte in a plant, comprising: (a) a measurement process comprising placing the regenerative plant sensor on a plant; applying a first electrical potential to the one or more SMA wires embedded in the flexible substrate to open the fluid gate allowing plant sap to flow over the sensor layer; applying a second electrical potential to the one or more SMA wires embedded in the flexible substrate to close the fluid gate; measuring a signal corresponding to an amount of analyte in the plant sap; and (b) a regeneration process comprising pumping the regeneration solution over the sensor layer via the microfluidic system to regenerate the sensor layer; and removing the regeneration solution from the sensor layer by pumping the regeneration solution away from the sensor layer via the microfluidic system to complete a measurement cycle.

In some aspects, the plant may be a sugar cane. In some aspects, the regenerative plant sensor may be placed on the stalk of the plant. In some aspects, the method may comprise measuring the amount of sucrose in the plant sap. In some aspects, wherein a sugar cane maturity may be determined by the amount of sucrose. In some aspects, a sugar cane harvest time may be determined by the amount of sucrose. In some aspects, the method may comprise measuring the amount of a plant hormone in the plant sap.

To prevent the plant from rejecting the sensor after insertion and to avoid the formation of an air gap between the sensor surface and plant tissue, the DTBA derivative is combined with a polyvinyl alcohol (PVA)-based hydrogel. Because of their high water content, hydrogels are flexible and have tissue-like mechanical properties. Hence, the combined formulation of hydrogel and DTBA derivative keeps the sensor surface moist and similar to plant tissue.

The sensor can measure sucrose concentrations of, for example, between about 1% and about 5%, between about 5% and about 10%, between about 15% and about 25%, or between about 50 mM and about 150 mM, between about 200 mM and about 500 mM, between about 100 mM and about 250 mM, or between about 150 mM and about 750 mM. Furthermore, the assay layer achieves a sucrose resolution of about 0.1%, about 0.3%, about 0.5%, about 0.8%, about 1.0%, or about 2.0%, or about 10 mM, about 20 mM, about 30 mM, about 50 mM, about 100 mM, or about 150 mM.

In some aspects, the ability of the sensor for measuring a signal corresponding to an amount of analyte in the plant sap is fully restored after a regeneration. In some aspects, the ability of the sensor for measuring a signal corresponding to an amount of analyte in the plant sap is superior after a measurement cycle compared to the ability of a sensor for measuring a signal corresponding to an amount of analyte in the plant sap which was not washed with regeneration solution. In some aspects, the ability of the sensor for measuring a signal corresponding to an amount of analyte in the plant sap is fully restored after a measurement cycle. In some aspects, the regeneration process improves one or more of the stability, reproducibility, repeatability, sensor drift, or signal-to-noise ratio of the sensor for measuring a signal corresponding to an amount of analyte in the plant sap in the method compared to a method which does not include a regeneration process.

In some aspects, a detection range for the sucrose is from about 0 mM to about 1,000 mM, about 1 mM to about 900 mM, about 100 mM to about 800 mM, or about 150 mM and about 750 mM.

In some aspects, an analyte detection sensitivity is about 90% to about 100%, about 91%, 92%, 93%, 94%, 95%, 96%, 97%, 98%, or 99% after at least one measurement cycle. In some aspects, an analyte detection sensitivity is about 90% to about 100%, about 91%, 92%, 93%, 94%, 95%, 96%, 97%, 98%, or 99% after two or more measurement cycles or three or more measurement cycles. In some aspects, an analyte detection sensitivity is about 100% after the measurement cycle.

In some aspects, the regeneration process of (b) increases a detection range for the amount of analyte in the plant sap compared to a method which does not include the step (b). In some aspects, the regeneration process of (b) decreases variability of the signal corresponding to an amount of analyte in the plant sap compared to a method which does not include the step (b). In some aspects, the regeneration process of (b) reduces a signal-to-noise ratio compared to a method which does not include step (b).

The fabrication method was achieved with the exclusion of bioagents, such as enzymes, aptamers, or antibodies. Bioagents can compromise sensor stability, shelf life, and reusability, and often exhibit variability with temperature and pH changes. Sensors based on biological compounds exhibit drift over time, causing inaccurate measurements. This can be due to changes in the compound's composition or its interaction with the target sample. By using non-enzymatic coatings and excluding bioagents, the sensor achieves greater stability and a longer shelf life.

In some embodiments, the present disclosure includes any one or combination of the following non-limiting numbered items:

The present application also provides aspects as set forth in the following numbered statements:
1. A regenerative plant sensor, comprising:
   a flexible substrate embedded with one or more shape memory alloy (SMA) wires,
   a substrate comprising a sensor layer, the sensor layer comprising a working electrode (WE), a counter electrode (CE), and a reference electrode (RE), wherein one or more electrodes on said sensor layer are functionalized with one or more of a first metal, a second metal, and a composition comprising a synthetic polymer and a hydrogel,
   a microfluidic system in communication with a fluid chamber and the sensor layer, one or more pumps in communication with the microfluidic system,
   a microcontroller in electrical communication with the sensor layer, the one or more pumps, and the one or more shape memory alloy (SMA) wires,
   wherein the one or more SMA wires form a fluid gate in the flexible substrate, and
   wherein the fluid chamber is filled with a regeneration solution.
2. The regenerative plant sensor of item 1, wherein the WE and CE are functionalized with a sputter-coated layer of the first metal.
3. The regenerative plant sensor of item 1 or item 2, wherein the WE is further functionalized with an electrically deposited layer of the first metal.
4. The regenerative plant sensor of any preceding item, wherein the first metal is gold.
5. The regenerative plant sensor of any preceding item, wherein RE is functionalized with a layer of the second metal.
6. The regenerative plant sensor of item 5, wherein the second metal is silver and/or silver chloride.
7. The regenerative plant sensor of any preceding item, wherein the WE is further functionalized with an assay layer
8. The regenerative plant sensor of item 7, wherein the assay layer comprises the composition comprising a synthetic polymer and a hydrogel.
9. The regenerative plant sensor of any preceding item, wherein the synthetic polymer interacts with a sugar or a hormone.
10. The regenerative plant sensor of any preceding item, wherein the synthetic polymer is di-thio-butyric acid (DTBA) or a derivative thereof.
11. The regenerative plant sensor of any one of items 9-10, wherein the sugar is sucrose.
12. The regenerative plant sensor of any preceding item, wherein the hydrogel is a polyvinyl alcohol (PVA)-based hydrogel or a derivative thereof.
13. The regenerative plant sensor of any preceding item, wherein the fluid gate is closed in a first state.
14. The regenerative plant sensor of any preceding item, wherein the fluid gate is open in second state.
15. The regenerative plant sensor of any preceding item, wherein the one or more SMA wires comprise nickel and titanium.
16. The regenerative plant sensor of any preceding item, wherein the one or more SMA wires comprise at least one one-way actuated wire and at least one two-way actuated wire.
17. The regenerative plant sensor of any one of the preceding items, wherein the regeneration solution comprises an acetate buffer.
18. The regenerative plant sensor of item 17, wherein the acetate buffer is at a concentration between about 1 mM to about 15 mM.
19. The regenerative plant sensor of item 18, wherein the acetate buffer is at a concentration of about 1 mM
20. The regenerative plant sensor of any one of items 17-19, wherein the regeneration solution has a pH of between about 4 to about 5.
21. The regenerative plant sensor of item 20, wherein the regeneration solution has a pH of about 4.5
22. The regenerative plant sensor of any preceding item, wherein the flexible substrate is a thermoplastic and/or thermosetting resin.
23. The regenerative plant sensor of any preceding item, wherein the thermoplastic and/or thermosetting resin is polydimethylsiloxane or a silicone rubber.
24. The regenerative plant sensor of any preceding item, wherein the flexible substrate has a thickness of about 50 μm to about 500 μm, about 100 μm to about 400 μm, about 50 μm to about 1000 μm, about 100 μm to about 1500 μm, or about 50 μm to about 2000 μm.
25. The regenerative plant sensor of any preceding item, wherein the flexible substrate comprises a polymer material.
26. The regenerative plant sensor of any preceding item, wherein the polymer material is flexible polydimethylsiloxane or polybutylene adipate terephthalate (PBAT).
27. The regenerative plant sensor of any preceding item, wherein the sensor layer is bioagent-free.
28. The regenerative plant sensor of any preceding item, further comprising a data acquisition system, wherein the data acquisition system comprises a processor; a communication unit; a memory unit; and wherein the data acquisition system is in communication with the sensor layer.
29. The regenerative plant sensor of item 28, wherein the memory unit is communicatively coupled to the processor, the memory unit having stored thereon computer software comprising a set of instructions that, when executed by the processor, causes the data acquisition unit to receive sensor data from the one or more sensors; and send, via the communication unit, the sensor data to an external device.
30. The regenerative plant sensor of item 28 or item 29, wherein the memory unit comprises a non-transitory computer-readable medium.
31. The regenerative plant sensor of item 29, wherein the sensor data are sent, via the communication unit, to an Internet of Things (IoT) cloud server configured to interact with one or more IoT-capable devices.
32. The regenerative plant sensor of any preceding item, further comprising a power supply unit.
33. The regenerative plant sensor of any one of items 30-32, wherein the microcontroller and the data acquisition system each comprise a power supply unit.
34. The regenerative plant sensor of item 29, wherein the instructions comprise a signal calibration.
35. The regenerative plant sensor of any preceding item, wherein the regenerative plant sensor has a total volume of about 200 to about 1,000 cm³, about 300 to about 800 cm³, or about 400 to about 600 cm³.
36. A method of measuring an analyte in a plant, comprising:
   (a) a measurement process comprising placing the regenerative plant sensor according to any one of items 1-35 on a plant;
   applying a first electrical potential to the one or more SMA wires embedded in the flexible substrate to open the fluid gate allowing plant sap to flow over the sensor layer;
   applying a second electrical potential to the one or more SMA wires embedded in the flexible substrate to close the fluid gate;
   measuring a signal corresponding to an amount of analyte in the plant sap; and
   (b) a regeneration process comprising pumping the regeneration solution over the sensor layer via the microfluidic system to regenerate the sensor layer; and
   removing the regeneration solution from the sensor layer by pumping the regeneration solution away from the sensor layer via the microfluidic system;
   wherein (a) and (b) constitute a measurement cycle.
37. The method of item 36, wherein the step of applying the first electrical potential opens the SMA wire gate in about 10 to about 60 seconds or about 15 to about 30 seconds.
38. The method of item 36 or item 37, wherein the step of applying the second electrical potential closes the SMA wire gate in about 10 to about 60 seconds or about 15 to about 30 seconds.
39. The method of any one of items 36-38, wherein the analyte is measured for about 1 to about 5 minutes or about 2 to about 3 minutes.
40. The method of any one of items 36-39, wherein the data acquisition system processes the data from the sensor layer to produce a data output.
41. The method of item 40, wherein the processed data are stored in the memory unit.
42. The method of any one of items 40 to 41, wherein the processed data are sent, via the communication unit, to the IoT cloud server configured to interact with the one or more IoT-capable devices.
43. The method of any one of items 36-42, wherein the regeneration solution is in contact with the sensor surface for about 1 minute to about 10 minutes, about 2 minutes to about 8 minutes, about 3 minutes to about 6 minutes, or about 4 minutes to about 5 minutes.
44. The method of any one of items 36-43, wherein the regeneration solution is removed from the sensor layer in about 10 to about 60 seconds, about 10 to about 30 seconds, or about 15 to about 20 seconds.
45. The method of any one of items 36-44, wherein the measurement cycle is completed in about 2 minutes to about 20 minutes, about 3 minutes to about 12 minutes, or about 5 minutes to about 10 minutes.
46. The method of any one of items 36-45, wherein the amount of analyte in the plant sap is sent to the IoT capable device.
47. The method of any one of items 36-46, wherein the analyte is about 100 mM to about 1,000 mM or about 150 mM to about 750 mM.
48. The method of any one of items 36-47, wherein the analyte is about 1% to about 40% or about 5% to about 25% of the plant sap.
49. The method of any one of items 36-48, wherein the signal corresponding to an amount of analyte in the plant sap is measured at a resolution of about 10 mM to about 100 mM, about 20 mM to about 200 mM, or about 10 mM to about 50 mM.
50. The method of any one of items 36-49, wherein the signal corresponding to an amount of analyte in the plant sap is measured at a resolution of about 0.015% to about 1.5%, about 0.1% to about 1%, or about 0.25% to about 0.75%.
51. The method of any one of items 36-50, wherein the total power consumption for the measurement cycle is about 1 W to about 15 W or about 2 W to about 8 W.
52. The method of any one of items 36-51, wherein the power supply unit supplies power for about 5 measurement cycles to about 100 measurement cycles, about 10 measurement cycles to about 75 measurement cycles, about 20 measurement cycles to about 60 measurement cycles, or about 30 measurement cycles to about 50 measurement cycles.
53. The method of any one of items 36-52, wherein the analyte is measured every 3 minutes to about 25 minutes, about 5 minutes to about 15 minutes, or about 7 minutes to about 10 minutes.
54. The method of any one of items 36-53, wherein the regenerative plant sensor is placed on the stalk of the plant.
55. The method of item 54, wherein a detection range for the analyte is from about 0 mM to about 1,000 mM, about 1 mM to about 900 mM, about 100 mM to about 800 mM, or about 150 mM and about 750 mM.
56. The method of any one of items 36-55, wherein an analyte detection sensitivity is about 90% to about 100%, about 91%, 92%, 93%, 94%, 95%, 96%, 97%, 98%, or 99% after at least one measurement cycle.
57. The method of any one of items 56, wherein an analyte detection sensitivity is about 90% to about 100%, about 91%, 92%, 93%, 94%, 95%, 96%, 97%, 98%, or 99% after two or more measurement cycles or three or more measurement cycles.
58. The method of any one of items 36-57, wherein an analyte detection sensitivity is about 100% after the measurement cycle.
59. The method of any one of items 36-58, wherein the regeneration process of (b) increases a detection range for the amount of analyte in the plant sap compared to a method which does not include the step (b).
60. The method of any one of items 36-59, wherein the regeneration process of (b) decreases variability of the signal corresponding to an amount of analyte in the plant sap compared to a method which does not include the step (b).
61. The method of any one of items 36-60, wherein the regeneration process of (b) reduces a signal-to-noise ratio compared to a method which does not include step (b).
62. The method of any one of items 36-61, wherein the plant is a sugar cane.
63. The method of any one of items 36-61, wherein the analyte is a plant hormone.
64. The method of any one of items 36-63, wherein the analyte is sucrose.
65. The method of any one of items 54-64, wherein a sugar cane maturity is determined by the amount of sucrose.
66. The method of any one of items 54-65, wherein a sugar cane harvest time is determined by the amount of sucrose.
67. A method of producing a regenerative plant sensor comprising:
   a) 3d printing a substrate comprising a sensor layer comprising a reference electrode (RE), a working electrode (WE) and a counter electrode (CE),
   b) plasma treating the substrate,
   c) depositing a titanium adhesion layer onto the sensor layer of the substrate, d) depositing a gold layer onto the WE and the CE,
   e) coating the RE with Ag/AgCl ink, and
   f) bonding wires to the electrodes.
68. The method of item 67, wherein step a) comprises 3d printing the substrate with a stereolithography (SLA) printer.
69. The method of item 67 or 68, wherein the titanium adhesion layer is about 10 nm thick.
70. The method of any one of items 67-69, wherein step d) comprises depositing the gold layer via sputtering.
71. The method of item 70, wherein the gold layer is about 200 nm thick.
72. The method of any one of items 67-71, wherein step f) comprises bonding the wires to the electrodes using silver ink and/or adhesive glue.
73. The method of any one of items 67-72, further comprising electrodepositing gold onto the WE and/or the CE.
74. The method of item 73, wherein the electrodeposition is performed using cyclic voltammetry in a gold (III) chloride trihydrate solution.
75. The method of any one of items 67-74, wherein after step a) and before step b), the method further comprises patterning a WE and a CE masking.
76. The method of item 75, wherein the masking is patterned with a vinyl cutter.
77. A method of producing a boronic assay solution, comprising:
   a) preparing a first solution comprising 3-aminophenyl boronic acid hemi sulfate salt (3-PBA) at a pH of 4-6,
   b) preparing a linker solution comprising 1-ethyl-3-(3-dimethylaminopropyl) carbodiimide hydrochloride (EDC-HCl),
   c) preparing a second solution comprising 4,4′-dithio-3-di(n-butyric acid) (DTBA) at a pH of 4-6,
   d) mixing the first solution with the linker solution to form a first mixture,
   e) adding the first mixture to the second solution to form a reaction mixture,
   f) incubating the reaction mixture at 4° C. for about 8-16 hours, then centrifuging to sediment an assay slurry, and
   g) isolating the assay slurry, then reconstituting the assay slurry in a methanol:water solution to form the boronic assay solution.
78. The method of item 77, wherein the first solution comprises about 5 mM 3-PBA.
79. The method of item 77 or 78, wherein the first solution has a pH of about 4.8.
80. The method of any one of items 77-79, wherein the first solution is maintained at about 4° C.
81. The method of any one of items 77-80, wherein the linker solution comprises about 5 mM EDC-HCl.
82. The method of any one of items 77-81, wherein step b) further comprises incubating the linker solution at about 0° C. for about 30 minutes.
83. The method of any one of items 77-82, wherein the second solution comprises about 2 mM DTBA.
84. The method of any one of items 77-83, wherein the second solution has a pH of about 4.8.
85. The method of any one of items 77-84, wherein the second solution is maintained at about 4° C.
86. The method of any one of items 77-85, wherein step e) further comprises stirring the reaction mixture for about 2 hours.
87. The method of any one of items 77-86, wherein step f) comprises centrifuging the reaction mixture at about 3500 rpm for about 2 hours.
88. The method of any one of items 77-87, wherein the methanol:water solution has a v:v ratio of 1:3.
89. A boronic assay solution produced by the method of any one of items 77-88.
90. A regenerative plant sensor produced by the method of any one of items 67-76.
91. A method of producing a sucrose sensor, comprising incubating at least a portion of the regenerative plant senor of item 90 with the boronic assay solution of item 89.
92. A method of processing electrodes a regenerative plant sensor comprising:
a) masking a working electrode (WE) and a counter electrode (CE),
b) plasma treating the masked electrodes,
c) depositing a titanium adhesion layer onto the WE and CE,
d) depositing a gold layer onto the WE and the CE,
e) coating a reference electrode (RE) with silver ink, and
f) bonding wires to the electrodes.

### EXAMPLES

The following examples are intended to exemplify the present disclosures and are not limitations of the claimed invention. All molecules, compositions, methods, assays, and results disclosed in the examples form part of the present invention.

### Example 1: Sensor Preparation

Step 1: Electrode preparation. The sensor fabrication process included 3D printing the substrate using a stereolithography (SLA) printer. Next, the masking was patterned with a vinyl cutter, followed by plasma cleaning to enhance hydrophilicity. Next, a 10 nm-thick titanium adhesion layer and a 200 nm-thick gold layer were deposited via sputtering. To further enhance the electrode surface, gold electrodeposition was performed using cyclic voltammetry in a gold (III) chloride trihydrate solution, which increases the surface area and improves sensor repeatability. The reference electrode was coated with silver/silver chloride ink, finalizing the electrode preparation. Lastly, wire bonding was completed using silver ink and adhesive glue. The process flow is shown in FIG. 47A.

Step 2: Synthetic boronic assay preparation and incubation. Solution 1, linker, and solution 2 were prepared as described below then used to prepare boronic assay solution. The process is summarized in FIG. 47B.

Solution 1: prepare 5.0 mM concentrated 3-aminophenyl boronic acid hemi sulfate salt (3-PBA)—then bring it to the pH 4.8 with 0.1N NaOH—maintain at 4° C. in ice bath.

Linker: prepare 5.0 mM concentrated 1-Ethyl-3-(3-dimethylaminopropyl) carbodiimide-Hydrochloride-powder form in aqueous medium as linker (EDC-HCl)—maintain at 0° C. for 30 min.

Solution 2: prepare 2.0 mM concentrated 4.4′-Dithio-3-di (n-butyric acid) (DTBA)—then bring it to pH 4.8 with 0.1N NaOH—maintain at 4° C. in an ice bath.

Solution 1 was mixed with EDC-linker and stirred for proper mixing, then added slowly to solution 2. This reaction mixture was stirred for 2 hours in an ice beaker. After rigorous mixing, it was kept overnight at 4° C. for about 12 hours for the assay slurry to form. The obtained white sedimentation is boronic assay activated product, which was later centrifuged in equal volumes at 350 rpm for about 2 hours.

Final white crude boronic group activated assay slurry was reconstituted in methanol (MeOH:Water 1:3 ratio). Specifically, each 10 μL volume equivalent slurry was dissolved in 30 μL of diluted methanol. This reconstituted solution showed complete miscibility and as ready for sensor incubation. The sensor was incubated each time with 10 μL of reconstituted volumes of boronic assay solution (multiple volumes to form multiple layers), which takes about 24 hours for 2 layers.

Step 3: Sensor pre- and post-conditioning. For all field studies, the initially developed gold working electrode (WE) sensors were first evaluated for connection issues by performing Electrochemical Impedance Spectroscopy (EIS) measurements on the bare gold surface, alongside predefined open-circuit potential (OCP) values. In the second phase, the sensors were tested again at fixed OCPs after incubating the boronic assay on the gold W.E. surfaces. This was done to ensure consistency in the EIS Nyquist plot signals by analyzing charge transfer resistance (Rct) values. The Nyquist plot was fitted using the Randles equivalent circuit model, incorporating solution phase resistance (Rs), constant phase element (CPE-Q), charge transfer resistance (Rct), and Warburg diffusion (W) at a 45-degree angle.

### Example 2

Reproducibility of the Regenerative Plant Sensor. Reproducibility of the regenerative plant sensor to measure sucrose was assessed. Nyquist Electrochemical Impedance Spectroscopy (EIS) was employed as a data collection technique, and measurements were taken daily over 16 days, with plots taken at each day (after day 1, after day 2, after day 3, etc.).

During each daily measurement, the sensor was subjected to alternating current signal within a frequency range of 0.1 Hz to 50 kHz, and impedance data was recorded across this spectrum.

Calibrated dose response (CDR) plots for the tests are shown in FIGS. 9-24. The Nyquist curve (in FIGS. 9A-24A) shows plots of imaginary part of impedance (Z″) versus the real part of the impedance (Z′). The correlation plot (at right, in FIGS. 9B-24B) shows a scatter plot of Rct versus sucrose dose. Rct refers to charge transfer resistance, which is derived from the Nyquist plot through an equivalent Randles circuit fitting. Next, a straight line is fitted through the scattered Rct values to get the CDR correlation plot.

### Example 3

Stability of the Regenerative Plant Sensor. Stability of the regenerative plant sensor to measure sucrose was assessed. A series of weekly measurements were taken over a period of 3 weeks to determine signal response at fixed sucrose doses. Sucrose doses of 150 mM (low) and 510 mM (high) were used to assess both sensitivity and signal range consistency.

At each of 3 weeks (week 1, week 2, week 3), the sensor layer was exposed to the selected sucrose concentrations, and the dose-response curves of FIGS. 25-26 were generated by plotting Rct (charge transfer resistance, in Ohms) against time for the low-dose (FIG. 25A) and high-dose (FIG. 26A). At right, data for the low-dose (FIG. 25B) and high-dose (FIG. 26B) are shown as Nyquist curves with plots of imaginary part of impedance (Z″) versus the real part of the impedance (Z′).

Data for the low-dose (150 mM) sucrose is shown in Table 1, below:

TABLE 1
Sucrose Sensor Assay Stability, 3 Weeks Results (Low Dose)

| | 150 mM Sucrose low-dose | | | | | |
|---|---|---|---|---|---|---|
| week-1 | 108522 | 108491 | 106991 | 104624 | 102860 | 101740 |
| week-2 | 96459 | 94019 | 94100 | 93049 | 91901 | 89942 |
| week-3 | 86287 | 86023 | 84588 | 82588 | 81185 | 81106 |

Data for the high-dose (510 mM) sucrose is shown in Table 2, below:

TABLE 2
Sucrose Sensor Assay Stability, 3 Weeks Results (High Dose)

| | 510 mM Sucrose low-dose | | | | | |
|---|---|---|---|---|---|---|
| week-1 | 77643 | 77284 | 76111 | 75900 | 74204 | 73091 |
| week-2 | 68993 | 69422 | 67982 | 65094 | 64910 | 63922 |
| week-3 | 61782 | 60700 | 58882 | 57286 | 56680 | 55985 |

### Example 4

Performance of the Regenerative Plant Sensor. Performance of the regenerative plant sensor to measure sucrose before regeneration was assessed via stability, repeatability and reproducibility experiments. The stability of the assay was assessed over three weeks, and reproducibility was considered at low (150 mM) and high (510 mM) doses. For each time point, the charge transfer resistance (Rct, in ohms) was recorded using EIS.

Stability data is shown in FIGS. 27A-27C. Data was collected at day 1, day 2, day 3, day 4, and day 5 for each of weeks 1, 2, and 3.

Reproducibility data is shown in FIGS. 28A-28B. Data was collected at each of weeks 1, 2, and 3.

### Example 5

Competitive Binding Assay. Ability of the regenerative plant sensor to regenerate was tested in acetate buffer at an acidic pH of about 4.5 to about 5.5. Competitive binding of sucrose to the DTBA PBA molecule is shown in FIG. 29.

As shown in FIG. 29, the acetate buffer breaks the sucrose bond with the DTBA-PBA assay by freeing two diol-OH reactive sites in the assay. This bond breakage occurs at an acidic pH (˜4.5) and at as a certain concentration of the acetate buffer (in the 1 mM to 10 mM range). As a result, the diol-OH reactive sites are available for new sucrose molecules.

It was determined that regeneration under the above-described conditions presented no time delay (that is, a complete regeneration run could be conducted in approximately 5 minutes), with no surface layer disturbance.

### Example 6

Sensor Regeneration at different pH values. Sucrose assay regeneration studies were conducted with different acetate buffer pH values, wherein the buffer concentration was held constant (10 mM).

Results of the competitive binding of acetate buffer vs diol at pH 5.5 (M1 sensor) are shown in FIG. 30. The DTB-PBA-Assay concentration was about 5 mM and the acetate buffer concentration was about 10 mM. The pH was adjusted to about 5.5. Data for this experiment is also presented in Table 3, below.

TABLE 3

| M1 sensor [Assay con: 5 mM | |
|---|---|
| Buffer con: 10 mM | |
| pH adjustment −5.5] | Layer Rct(KΩ) |
| DTBA-PBA-assay | 8462 |
| DTBA-PBA-assay | 8559 |
| Sucrose-first dose- 1000 mM | 6009 |
| Sucrose-first dose- 1000 mM | 6072 |
| Sucrose -sec dose-1000 mM | 5560 |
| Sucrose -sec dose-1000 mM | 5568 |
| acetate buffer @5.5 pH | 1875 |
| acetate buffer @5.5 pH | 4005 |
| acetate buffer @5.5 pH | 4000 |
| Assay regeneration | 7936 |
| Assay regeneration | 8033 |

Results of the competitive binding of acetate buffer vs diol at pH 4.5 (M2 sensor) are shown in FIG. 31. The DTB-PBA-Assay concentration was about 10 mM and the acetate buffer concentration was about 10 mM. The pH was adjusted to about 4.5. Data for this experiment is also presented in Table 4, below.

TABLE 4

| M2 sensor [Assay con: 10 mM | |
|---|---|
| Buffer con: 10 mM | |
| pH adjustment −4.5] | Layer Rct(KΩ) |
| DTBA-PBA-assay | 15196 |
| DTBA-PBA-assay | 14792 |
| Sucrose-first dose- 1000 mM | 12000 |
| Sucrose-first dose- 1000 mM | 11900 |
| Sucrose -sec dose-1000 mM | 10460 |
| Sucrose -sec dose-1000 mM | 10500 |
| Sucrose -third dose-1000 mM | 9981 |
| acetate buffer @4.0 pH | 9000 |
| acetate buffer @4.0 pH | 4747 |
| Assay regeneration | 14556 |
| Assay regeneration | 14716 |

Results of the competitive binding of acetate buffer vs Diol at pH 5.5 (M4 sensor) are shown in FIG. 32. The DTB-PBA-Assay concentration was about 10 mM and the acetate buffer concentration was about 10 mM. The pH was adjusted to about 5.5. Data for this experiment is also presented in Table 5, below.

TABLE 5

| M4 sensor[Assay con: 10 mM | |
|---|---|
| Buffer con: 10 mM | |
| pH adjustment −5.5] | Layer Rct(KΩ) |
| DTBA-PBA-assay | 20263 |
| DTBA-PBA-assay | 20100 |
| Sucrose 1000 mM | 12222 |
| Sucrose 1000 mM | 12205 |
| Sucrose 1000 mM | 11953 |
| Sucrose 1000 mM | 11900 |
| acetate buffer @5.5 pH | 7509 |
| acetate buffer @5.5 pH | 7467 |
| Assay regeneration | 19982 |
| Assay regeneration | 19551 |

### Example 7

Sensor Regeneration Buffer Concentration Assay. Sucrose assay regeneration studies were conducted with different acetate buffer concentrations, wherein the buffer pH was held constant (DTBA-PBA assay pH of 4.8, acetate buffer pH of 4.5) at low, medium, and high concentrations of sucrose.

At low concentrations of sucrose, such as between about 1 mM and about 150 mM, the acetate buffer was optimal to regenerate the assay surface. It was shown that the 1 mM acetate buffer could wash out at pH 4.5 and completely regenerate the sensor surface. However, highly concentrated acetate buffer (for example, about 10 mM) was found to be disturbing the assay layer.

Results of the assay using different buffer concentrations with low concentrations of sucrose are shown in FIG. 33.

Experiments were also carried out at medium concentrations of sucrose, about 510 mM. After a first sucrose dosing, the sensor was spiked 1 mM of acetate buffer. After a second sucrose dosing, the sensor was spiked 10 mM of acetate buffer. Sensor assay surface loss was observed after spiking with 10 mM of acetate buffer. It was determined that, for regeneration purposes, 1 mM buffer works well for moderate sucrose levels.

Results of the assay with different buffer concentrations with medium concentrations of sucrose are shown in FIG. 34.

Further experiments were carried out at high concentrations of sucrose, about 750 mM. At high concentrations, 10 mM acetate buffer was shown to be successful. It was determined that, if 1 mM of acetate buffer is used, multiple washes may be needed to regain the assay surface. Thus, acetate buffer concentrations in the range of between about 1 mM and about 10 mM at a pH of about 4.5 were evaluated using various sucrose concentrations ranging from between about 150 mM to about 750 mM, which is the range of sucrose concentrations found in sugarcane stalk.

Results of the buffer concentration assay with high concentrations of sucrose are shown in FIG. 35.

### Example 8

Field Test at USDA-ARS's Sugarcane Field in Houma, LA. Regenerative plant sensors were tested in the field at USDA-ARS's Sugarcane Field in Houma, LA. The regeneration washing process used is shown in FIGS. 36A-36B. The regeneration washing process included a regenerative solvent of 1 mM acetate buffer at pH 4.5.

Results of the regeneration field test are shown in FIG. 37. After drop-casting DI water on the sensor, electrochemical impedance spectroscopy (EIS) was conducted using a potentiostat (Emstat 4s) three times over a two-hour period. The assay readings are represented by red dots.

Next, filtered sugarcane juice with a Brix value of 18.3% was applied to the sensor, and an EIS reading was taken. The sensor surface was washed using a pump setup with a 1 mM regenerative acetate buffer solution for 30 seconds, repeated three times.

Following this, the sensor was rinsed with DI water to eliminate any remaining buffer solution, and an EIS reading (Assay reading-after regeneration) was recorded to compare with the assay readings taken without any sucrose dosing. This regeneration process was repeated three times, with the 1^{st}, 2^{nd}, and 3rd sucrose readings and post-regeneration assay readings displayed using blue, purple, and green dots, respectively.

A comparative analysis is shown in FIG. 38A-38B. As shown in FIG. 38A, known concentrations of sucrose solutions were manually deposited on the sensor surface (1 replicate, 3 different concentrations to cover the entire sucrose range). No nitinol or pump setup was used.

As shown in FIG. 38B, a full pump setup was used, and cane juice (3 replicates, one Brix value of 18.3) was deposited on the sensor.

The comparative analysis of FIG. 38A-38B demonstrates that the regenerative solvent (sodium acetate buffer at 1 mM and pH 4.5, aligned with the boronic assay pH of 4.8), along with multiple water washes, successfully restored or reactivated the assay layer.

Live sugarcane plant data is presented in FIG. 39A-39B. The regenerative plant sensors were mounted on live sugarcane plants. For comparison, the model 299 cane (Brix=16) plant (FIG. 39A) had a lower sucrose content than the model 508 (Brix=17.1) plant (FIG. 39B).

Sensor data recorded between two consecutive days revealed that the Ret difference (i.e., ΔRct) in the high-sucrose model 508 plant (FIGS. 39B, 43B) was considerably higher than in the model 299 plant (FIGS. 39A, 43A). The change in Rct correlates with the Brix. Larger ΔRct means higher sucrose content. Thus, the sensor (through ΔRct) can differentiate high sucrose clones (model 508) from low sucrose clones (model 299). The data collected corresponded to mega-ohm Rct values in the crude sap from the cane plants, captured in real-time. While multiple assay layers were deposited on the sensor surface, higher assay concentrations were still required for accurate plant monitoring due to the unfiltered sap containing other saccharides, its thicker consistency compared to lab-prepared sucrose solutions, and variations in sap density.

Data from a sensor as described herein (labeled “Example”) was compared to standard Brix measurements obtained by USDA-Agricultural Research Service at Houma, LA (labeled “Comparative Example”) or direct measurement from Atago Refractometer for determinations of Brix values and calculation of milli Molar (mM) sucrose in cane juice samples. Results are shown in Table 6, below.

TABLE 6

| | | milli Molar | mM | calculated | |
|---|---|---|---|---|---|
| HOUMA | Brix values | sucrose | sucrose | Brix with | direct Brix |
| cane juice | (Comparative | (Comparative | with sensor | sensor | from Atago |
| samples | Example) | Example) | (Example) | (Example) | Refractometer |
| 1 | 18.3 | 534.62 | 533.48 | 18.26 | 18.3 |
| 2 | 18.3 | 534.62 | 533.78 | 18.27 | 18.3 |
| 3 | 19.9 | 581.36 | 564.61 | 19.33 | 19.3 |
| 4 | 18.2 | 531.70 | 524.33 | 17.99 | 17.9 |
| 5 | 19.2 | 560.91 | 539.78 | 18.48 | 18.6 |
| 6 | 19.7 | 575.52 | 589.45 | 20.18 | 20.2 |
| 7 | 18.6 | 543.38 | 563.96 | 19.30 | 19.4 |
| 8 | 18.5 | 540.46 | 561.43 | 19.22 | 19.3 |
| 9 | 18.1 | 528.78 | 548.72 | 18.78 | 18.8 |
| 10 | 19.1 | 557.99 | 558.29 | 19.11 | 19.0 |

Data from a sensor as described herein (labeled “Example”) was compared to standard Brix measurements obtained by USDA-Agricultural Research Service at Houma, LA (labeled “Comparative Example”) for determinations of Brix values and calculation of mM sucrose in six cane cultivar samples (listed by model number, at far left). Results are shown in Table 7, below.

TABLE 7

| Cane | Brix | | | |
|---|---|---|---|---|
| cultivar | values | mM sucrose | | Brix |
| model | (Comparative | (Comparative | mM sucrose | values |
| number | Example) | Example) | (Example) | (Example) |
| 15-508 | 17.10 | 499.56 | 497.24 | 17.02 |
| 15-306 | 18.00 | 525.85 | 528.40 | 18.09 |
| 14-885 | 16.00 | 467.43 | 461.74 | 15.81 |
| 11-9344 | 15.80 | 461.58 | 461.84 | 15.81 |
| 09-804 | 17.50 | 511.25 | 513.81 | 17.59 |
| 01-299 | 16.00 | 467.43 | 468.15 | 16.02 |

Sensor regeneration was also tested in situ at the Houma, LA site, as shown in FIGS. 44A-45. As shown in FIG. 44A, known concentrations of sucrose solutions were used (1 replicate, 3 different concentrations to cover the entire sucrose range) and the charge transfer resistance (Rct, in ohms) was recorded using EIS for (a) the assay layer (at left; after a volume of 10 μL had been deposited); (b) after a sucrose dose (at a volume of 10 μL); and (c) after the assay layer had been regenerated. Similar experiments were conducted for cane juice (3 replicates, one Brix value of 18.3, shown in FIG. 44B).

As shown in FIGS. 44A-44B, the regenerative solvent, as described herein as part of the regeneration process, successfully restores or reactivates the assay layer.

Similar results are shown in FIG. 45, which shows the change in Rct for experiments conducted with cane juice, at a Brix value of 16.2. Data was collected before and after regeneration for assay reading (left) and sugarcane juice reading (right). These results further confirm the success of the sensor regeneration in allowing for accurate, repeat use.

### Example 8

Field Test at Sugarcane Field in Canal Point, FL. Regenerative plant sensors were tested in a sugarcane field in Canal Point, FL. 27 sensors were tested in 9 cane varieties (listed in Table 8), with 3 sensors installed per cane variety (FIG. 48A). The sensors were installed in the canes and data were collected over 10 days (FIG. 48B). These 9 varieties were chosen for their broad range of Brix values (11% to 25%) to validate the sensors' functionality and accuracy across varying sugar concentrations.

TABLE 8

| | | Raw Juice from Canal point | | | | |
|---|---|---|---|---|---|---|
| | | | Sensor | | | |
| | | Refractometer | BRIX | rel | abs | |
| Category | Clone/Variety | BRIX value | value | diff | diff | |
| CAT 50 | MQ724005 | 16.8 | 16 | 0.8 | 0.8 | |
| CAT 21 | LF65-3611 | 16.2 | 15.5 | 0.7 | 0.7 | |
| CAT 25 | BR97-1004 | 15.5 | 15.8 | −0.3 | 0.3 | |
| CAT 42 | CP06-3103 | 18.2 | 16.5 | 1.7 | 1.7 | |
| CAT 27 | CP04-1844 | 16.6 | 16.2 | 0.4 | 0.4 | |
| CAT 103 | CL77-0727 | 20.5 | 19.9 | 0.6 | 0.6 | |
| CAT 07 | US16-2021 | 21.1 | 20.8 | 0.3 | 0.3 | |
| CAT 52 | CPCL01-0877 | 18.9 | 18.5 | 0.4 | 0.4 | |
| CAT 16 | CP12-2195 | 18.5 | 18.3 | 0.2 | 0.2 | |
| | | | | 0.53 | 0.60 | mean |
| | | | | 0.54 | 0.46 | SD |

In total, about 225 Brix readings were collected. Sensor readings were compared with 4 different existing Brix measurement techniques, near infrared (NIR), micro-NIR (mNIR), refractometer, and high-performance liquid chromatography (HPLC). Sensor measurements align closely with refractometry-based Brix readings obtained by ARS scientists and farmers, showing a strong correlation with R²=0.93 (FIG. 49). Moreover, a maximum average difference of 1.68±2.45 and a minimum average difference of 0.67±0.45 were observed.

Raw cane juice samples from the 9 cane varieties were also brought from Canal Point, FL to University of Texas Tyler to run additional tests, including sensor regeneration. Regeneration analyses were conducted both with freshly collected cane juice samples and inside the live cane. In-cane sensor regeneration was demonstrated at 3 time points in a full day with 2 different cane models and Brix values were compared with standard refractometer readings. The results are shown in FIG. 50.

### OTHER EMBODIMENTS

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

Other aspects, advantages, and modifications are within the scope of the following claims.

What is claimed is:

1. A regenerative plant sensor, comprising:
a flexible substrate embedded with one or more shape memory alloy (SMA) wires;
a substrate comprising a sensor layer, the sensor layer comprising a working electrode (WE), a counter electrode (CE), and a reference electrode (RE), wherein one or more electrodes on said sensor layer are functionalized with one or more of a first metal, a second metal, and a composition comprising a synthetic polymer and a hydrogel;
a microfluidic system in communication with a fluid chamber and the sensor layer;
one or more pumps in communication with the microfluidic system;
a microcontroller in electrical communication with the sensor layer, the one or more pumps, and the one or more shape memory alloy (SMA) wires;
wherein the one or more SMA wires form a fluid gate in the flexible substrate, and
wherein the fluid chamber is filled with a regeneration solution.

2. The regenerative plant sensor of claim 1, wherein:
a) the WE and CE are functionalized with a sputter-coated layer of the first metal,
b) the WE is further functionalized with an electrically deposited layer of the first metal,
c) the first metal is gold,
d) the RE is functionalized with a layer of the second metal, optionally wherein the second metal is silver and/or silver chloride, and/or
e) the WE is further functionalized with an assay layer, optionally wherein the assay layer comprises the composition comprising a synthetic polymer and a hydrogel.

3. The regenerative plant sensor of claim 1, wherein:
a) the synthetic polymer interacts with a sugar or a hormone, optionally wherein the sugar is sucrose, or
b) the synthetic polymer is di-thio-butyric acid (DTBA) or a derivative thereof.

4. The regenerative plant sensor of claim 1, wherein the hydrogel is a polyvinyl alcohol (PVA)-based hydrogel or a derivative thereof.

5. The regenerative plant sensor of claim 1, wherein the fluid gate is closed in a first state.

6. The regenerative plant sensor of claim 1, wherein the fluid gate is open in second state.

7. The regenerative plant sensor of claim 1, wherein the one or more SMA wires comprise nickel and titanium.

8. The regenerative plant sensor of claim 1, wherein the one or more SMA wires comprise at least one one-way actuated wire and at least one two-way actuated wire.

9. The regenerative plant sensor of claim 1, wherein the regeneration solution comprises an acetate buffer, optionally wherein the acetate buffer is at a concentration between about 1 mM to about 15 mM.

10. The regenerative plant sensor of claim 9, wherein the regeneration solution has a pH of between about 4 to about 5.

11. The regenerative plant sensor of claim 1, wherein the flexible substrate is a thermoplastic and/or thermosetting resin, optionally wherein the thermoplastic and/or thermosetting resin is polydimethylsiloxane or a silicone rubber.

12. The regenerative plant sensor of claim 1, wherein the flexible substrate has a thickness of about 50 μm to about 500 μm, about 100 μm to about 400 μm, about 50 μm to about 1000 μm, about 100 μm to about 1500 μm, or about 50 μm to about 2000 μm.

13. The regenerative plant sensor of claim 1, wherein the flexible substrate comprises a polymer material.

14. The regenerative plant sensor of claim 1, wherein the polymer material is flexible polydimethylsiloxane or polybutylene adipate terephthalate (PBAT).

15. The regenerative plant sensor of claim 1, wherein the substrate comprising the sensor layer has a thickness of about 1 mm to about 4 mm or about 2 mm to about 3 mm.

16. The regenerative plant sensor of claim 1, wherein the substrate comprising the sensor layer comprises a hardened resin.

17. The regenerative plant sensor of claim 1, wherein the sensor layer is bioagent-free.

18. The regenerative plant sensor of claim 1, further comprising a data acquisition system, wherein the data acquisition system comprises a processor; a communication unit; a memory unit; and wherein the data acquisition system is in communication with the sensor layer, optionally wherein the memory unit is communicatively coupled to the processor, the memory unit having stored thereon computer software comprising a set of instructions that, when executed by the processor, causes the data acquisition unit to receive sensor data from the one or more sensors; and send, via the communication unit, the sensor data to an external device, optionally wherein the instructions comprise a signal calibration, and optionally further comprising a power supply unit.

19. The regenerative plant sensor of claim 18, wherein the memory unit comprises a non-transitory computer-readable medium and/or wherein the sensor data are sent, via the communication unit, to an Internet of Things (IoT) cloud server configured to interact with one or more IoT-capable devices.

20. The regenerative plant sensor of claim 18, wherein the microcontroller and the data acquisition system each comprise a power supply unit.

21. A method of measuring an analyte in a plant, comprising:
(a) a measurement process comprising placing the regenerative plant sensor according to claim 1 on a plant;
applying a first electrical potential to the one or more SMA wires embedded in the flexible substrate to open the fluid gate allowing plant sap to flow over the sensor layer;
applying a second electrical potential to the one or more SMA wires embedded in the flexible substrate to close the fluid gate;
measuring a signal corresponding to an amount of analyte in the plant sap; and
(b) a regeneration process comprising pumping the regeneration solution over the sensor layer via the microfluidic system to regenerate the sensor layer; and
removing the regeneration solution from the sensor layer by pumping the regeneration solution away from the sensor layer via the microfluidic system;
wherein (a) and (b) constitute a measurement cycle.

22. The method of claim 21, wherein:
a) the step of applying the first electrical potential opens the SMA wire gate in about 10 to about 60 seconds or about 15 to about 30 seconds,
b) the step of applying the second electrical potential closes the SMA wire gate in about 10 to about 60 seconds or about 15 to about 30 seconds,
c) the analyte is measured for about 1 to about 5 minutes or about 2 to about 3 minutes,
d) the data acquisition system processes the data from the sensor layer to produce a data output, optionally wherein the processed data are stored in the memory unit, and optionally wherein the processed data are sent, via the communication unit, to an Internet of Things (IoT) cloud server configured to interact with the one or more IoT-capable devices,
e) the regeneration solution is in contact with the sensor surface for about 1 minute to about 10 minutes, about 2 minutes to about 8 minutes, about 3 minutes to about 6 minutes, or about 4 minutes to about 5 minutes,
f) the regeneration solution is removed from the sensor layer in about 10 to about 60 seconds, about 10 to about 30 seconds, or about 15 to about 20 seconds,
g) the measurement cycle is completed in about 2 minutes to about 20 minutes, about 3 minutes to about 12 minutes, or about 5 minutes to about 10 minutes,
h) the amount of analyte in the plant sap is sent to the IoT capable device,
i) the analyte is about 100 mM to about 1,000 mM or about 150 mM to about 750 mM,
j) the analyte is about 1% to about 40% or about 5% to about 25% of the plant sap,
k) the signal corresponding to an amount of analyte in the plant sap is measured at a resolution of about 10 mM to about 100 mM, about 20 mM to about 200 mM, or about 10 mM to about 50 mM,
l) the signal corresponding to an amount of analyte in the plant sap is measured at a resolution of about 0.015% to about 1.5%, about 0.1% to about 1%, or about 0.25% to about 0.75%,
m) the total power consumption for the measurement cycle is about 1 W to about 15 W or about 2 W to about 8 W,
n) a power supply unit supplies power to the regenerative plant sensor for about 5 measurement cycles to about 100 measurement cycles, about 10 measurement cycles to about 75 measurement cycles, about 20 measurement cycles to about 60 measurement cycles, or about 30 measurement cycles to about 50 measurement cycles,
o) wherein the analyte is measured every 3 minutes to about 25 minutes, about 5 minutes to about 15 minutes, or about 7 minutes to about 10 minutes,
p) the regenerative plant sensor is placed on the stalk of the plant,
q) a detection range for the analyte is from about 0 mM to about 1,000 mM, about 1 mM to about 900 mM, about 100 mM to about 800 mM, or about 150 mM and about 750 mM,
r) an analyte detection sensitivity is about 90% to about 100%, about 91%, 92%, 93%, 94%, 95%, 96%, 97%, 98%, or 99% after at least one measurement cycle,
s) an analyte detection sensitivity is about 90% to about 100%, about 91%, 92%, 93%, 94%, 95%, 96%, 97%, 98%, or 99% after two or more measurement cycles or three or more measurement cycles,
t) an analyte detection sensitivity is about 100% after the measurement cycle,
u) the regeneration process of (b) increases a detection range for the amount of analyte in the plant sap compared to a method which does not include the step (b),
v) the regeneration process of (b) decreases variability of the signal corresponding to an amount of analyte in the plant sap compared to a method which does not include the step (b),
w) the regeneration process of (b) reduces a signal-to-noise ratio compared to a method which does not include step (b),
x) the plant is a sugar cane, and/or
y) the analyte is a plant hormone or sucrose and a sugar cane maturity is determined by the amount of sucrose and a sugar cane harvest time is determined by the amount of sucrose.
